(19) United States
(12) Patent Application Publication
(10) Pub. No.: US 2017/0216219 A1
(43) Pub. Date: Aug. 3, 2017

(54) THERAPEUTIC NANOPARTICLES FOR ACCUMULATION IN THE BRAIN

(71) Applicant: UNIVERSITY OF GEORGIA RESEARCH FOUNDATION, INC., Athens, GA (US)

(72) Inventors: Shanta Dhar, Miami, FL (US); Emily Wyatt Baker, Athens, GA (US); Sean Marrache, Portland, OR (US); Franklin D. West, Bogart, GA (US)

(21) Appl. No.: 15/501,022

(22) PCT Filed: Aug. 3, 2015
(86) PCT No.: PCT/US15/43398

§ 371 Date: Feb. 1, 2017

Related U.S. Application Data
(60) Provisional application No. 62/032,828, filed on Aug. 4, 2014.

(51) Int. Cl. A61K 9/51; A61K 47/02; A61K 31/573; A61K 49/00; A61K 9/00; A61K 31/122

(52) U.S. Cl. CPC A61K 9/5153; A61K 9/0019; A61K 31/122; A61K 31/573; A61K 49/0067; A61K 47/02; A61K 47/48192; A61K 47/48953

(57) ABSTRACT

Nanoparticles containing a mitochondrial that are capable of crossing the blood-brain barrier and that have a targeting moiety, an antioxidant and an anti-inflammatory agent may be useful for treatment of traumatic brain injury.

### RELATED APPLICATION

This application claims the benefit of U.S. Provisional Patent Application No. 62/032,828, filed on Aug. 4, 2014, which application is hereby incorporated herein by reference in its entirety to the extent that it does not conflict with the disclosure presented herein.

### STATEMENT OF GOVERNMENTAL SUPPORT

This invention was made with government support under grant number P30GM092378, awarded by the National Institutes of Health of the United States government. The government has certain rights in the invention.

### FIELD

The present disclosure relates to nanoparticles configured to accumulate in the brain and methods of use thereof, including diagnostic and therapeutic uses. In some embodiments, the nanoparticles traffic agents such as antioxidants, anti-inflammatory agents, or both to mitochondria and may be used for treating damaged brain tissue, such as for treatment of traumatic brain injury.

### SUMMARY

The present disclosure describes, among other things, nanoparticles for delivering therapeutic agents or imaging agents across the blood-brain barrier and accumulation in the brain. The nanoparticles may include a mitochondrial targeting moiety to traffic the agents to mitochondria, particularly to mitochondria rich cells or regions of the central nervous system. The therapeutic agents may include antioxidants, anti-inflammatory agents, or both antioxidants and anti-inflammatory agents. The therapeutic agents may be used to treat disorders of the CNS. In some embodiments, the therapeutic nanoparticles are used to treat damaged CNS tissue, such as damaged neural tissue. In some embodiments, the therapeutic nanoparticles are used to treat traumatic brain injury and stroke. Imaging agents may include an imaging agent like QD, iron oxide, gadolinium or other clinically relevant imaging molecules which can be encapsulated in, attached to, or encapsulated in and attached to the nanoparticles.

Advantages of one or more of the various embodiments presented herein over prior nanoparticles, treatment modalities, or the like will be readily apparent to those of skill in the art based on the following detailed description when read in conjunction with the accompanying drawings.

### BRIEF DESCRIPTION OF THE DRAWINGS

FIG. 1A is a schematic drawing illustrating the design and construction of mitochondria-targeted (T) and non-targeted (NT) nanoparticles (NPs) for delivery of antioxidant and anti-inflammatory agents to the brain. Structures of polymers and payloads used and visual representation of targeted and non-targeted nanoparticles are depicted.

FIG. 1B is a number of graphs and images showing diameter (Z_{average}), zeta potential, loading, EE, and transmission electron micrographs of targeted and non-targeted NPs containing CoQ₁₀ and prednisone.

FIG. 2 is a number of graphs and images showing accumulation of T-NPs in the brain of normal Pig and distribution in brain white matter. American Landrace piglets (4 weeks old, n=3) were anesthetized using isofluorane. TQD-NPs and NT-QD-NPs were administered via ear vein IV. (A) Plasma circulation and different organ distribution of T and NT-NPs by ICP-MS in pig model. ***P<0.001. (B) 24 h post injection brain accumulation using IVIS, top: Normal photograph and bottom: Fluorescence image. (C) Distribution of T-NPs in the white matter of the brain by IVIS analyses of coronal slices of whole brain; top: normal photograph showing the white and grey matter; bottom: fluorescence images; and right side: quantitative analyses of white and grey matters by ICP-MS, data represents average from three piglets.

FIG. 3 is a number of images showing accumulation of T-NPs in the mitochondria of pig brain tissue. Grey and white matter samples were isolated from each brain. Brain samples were fixed and stained with MitoTracker green for mitochondria labeling and ProLong® Gold mounting media with DAPI for nuclear stain. (A) T-NPs showed significant co-localization with MitoTracker dye in both grey and white matter, (B) NT-NPs showed limited presents in grey and white matter brain tissues and no colocalization to the mitochondria. Scale bar: 25 μm.

FIG. 4 is a number of images and graphs showing changes associated with pig brain after TBI. (A) Neuronal death, neuroparenchymal edema, and neutrophil invasion after TBI. (I) The lesion site (blue arrow) is filled with hemorrhage and the white matter along the cortical junction is pale and rarified (p) due to edema. (II) Note lack of changes on the contralateral side. (III) A 40× magnification of the smaller box in (I). Neuronal cell death, characterized by shrunken red neuronal cell bodies and pyknotic nuceli, is present in the grey matter region of the lesion site that is not seen in a comparable area on the contralateral side (IV). (V) A 20× magnification of the affected grey matter. Neutrophils are not present in a comparable area of the contralateral side, and neuronal cell bodies are obvious (VI). (VII) A 40× magnification of the larger box in (I). Hemorrhage (h) is present in the white matter of the lesion site and the surrounding white matter is highly vacuolated (v) due to edema accumulation that is not present in a comparable area on the contralateral side (VIII). (I, II) scale bars=2 mm; (V, VI) scale bars=200 μm; (III, IV, VII, VIII) scale bars=100 μm. (B) Distinct volumetric changes due to edema accumulation on the ipsilateral side of injury. (Top) Coronal sections of piglet brain show lesion sites (blue arrow) with white matter regions of the ipsilateral side appearing swollen (black arrows) relative to comparable regions of the contralateral side (black arrowheads). (Bottom) The ipsilateral hemisphere was larger in area than the contralateral hemisphere due to swelling with an average increase of 13%. (C) Increased ROS levels after TBI. (D) Increased inflammatory cytokine levels after TBI. Both IFN-γ (top) and TNF-α (bottom) protein levels were significantly increased in injured brain tissues compared to uninjured brain tissues. n>3 for each treatment group.

FIG. 5 is a number of graphs and images showing accumulation of T-NPs in the brain of injured Pig. American Landrace piglets (4 weeks old) were anesthetized using isofluorane and the TBI was induced. After 5 h, T-QD-NPs and NT-QD-NPs were administered via ear vein i.v. (A) Plasma circulation of T-QD-NPs and NT-QD-NPs in TBI pig model. Brain accumulation was followed by ICP-MS (B) and (C) IVIS 24 h post injection. ***P<0.001; **P=0.001-0.01; ns=non-significant.

FIG. 6 is a number of graphs showing therapeutic potential of mitochondria targeted NPs carrying an antioxidant and an anti-inflammatory agent in NSCs. (A) Release kinetics of anti-inflammatory prednisone and antioxidant CoQ₁₀ from mitochondria targeted T-NPs and non-targeted NT-NPs. (B) Antioxidative properties of T-CoQ10-NPs and NT-CoQ10-NPs in NSC cells using Seahorse analyzer. (C) Anti-inflammatory properties of mitochondria targeted NPs carrying CoQ₁₀ and prednisone in NSCs. Cells were first treated with LPS (100 ng/mL) for 36 h. T-CoQ₁₀-NPs, NT-CoQ₁₀-NPs, T-Pred-NPs, NT-Pred-NPs, T-CoQ₁₀-NPs+T-Pred-NPs, NT-CoQ₁₀-NPs+NT-Pred-NPs, free CoQ₁₀, free prednisone, or free prednisone+free CoQ₁₀ (1 μM with respect to CoQ₁₀ or prednisone for all test articles) were added to LPS treated cells. ELISA was performed on the supernatants against TNF-#, IL-4, IL-6, and IL-10. The data represent the mean±S.D. ***, p<0.001; **, p=0.001-0.01; *, p=0.01-0.05; ns=non-significant.

FIG. 7 is a number of graphs and images showing dose dependent 14-day toxicity study in piglets. Saline or T-NPs or NT-NPs (two different doses, 5 mg/kg and 10 mg/kg with respect to total NP) were administered by intravenous injection and toxicity was followed for 14 days. (A) Complete serum chemistry results day 7 and day 14 after single intravenous injection of T-NPs, NT-NPs with 5 mg/kg and 10 mg/kg, and saline. (B) Representative images from day 14 post-injection histopathology of brain and liver from treated animals. No significant changes related to the T-NP or NT-NP injection were observed.

FIG. 8 is graphic timeline and graph illustrating therapeutic potential of mitochondria targeted NPs carrying an antioxidant and an anti-inflammatory agent in piglet model of TBI. (A) Schematic showing induction of TBI in piglets and administration of NPs. (B) Increased ROS levels after TBI and subsequent reduction in oxidative stress level in piglets treated with a combination of T-CoQ10 and T-Pred-NPs. The data represent the mean±S.D. ***, p<0.001; **, p=0.001-0.01; *, p=0.01-0.05; ns=non-significant.

FIG. 9 is a number of graphs showing overlay of DLS plots of diameter of T-Pred-NP and T-CoQ₁₀-NP (left) and diameter of NT-Pred-NP and NT-CoQ₁₀-NP (right).

FIG. 10 is a number of images showing IVIS analyses of the vertically cut slices of whole brain of American Landrace piglets (4 weeks old). The piglets were anesthetized using isofluorane and saline was administered via ear vein IV.

FIG. 11 is a number of images showing IVIS analyses of the vertically cut slices of whole brain of American Landrace piglets (4 weeks old). The piglets were anesthetized using isofluorane and NT-QD-NP was administered via ear vein IV.

FIG. 12 is a number of images showing IVIS analyses of the vertically cut slices of whole brain of American Landrace piglets (4 weeks old). The piglets were anesthetized using isofluorane and T-QD-NP was administered via ear vein IV.

FIG. 13 is a number of images showing IVIS analyses of whole brain and vertically cut slices of whole brain of American Landrace piglets (4 weeks old). The piglets were anesthetized using isofluorane and NPs (T-QD-NPs: 2.5 mg/kg with respect to NP and 0.46 mg/kg with respect to Cd); NT-QD-NPs: 2.5 mg/kg with respect to NP and 0.62 mg/kg with respect to Cd),) were administered via ear vein IV. The data show the images of all the animals from each group.

FIG. 14 is a number of images. American Landrace piglets (4 weeks old) were anesthetized using isofluorane and NPs (T-QD-NPs: 2.5 mg/kg with respect to NP and 0.46 mg/kg with respect to Cd); NT-QD-NPs: 2.5 mg/kg with respect to NP and 0.62 mg/kg with respect to Cd),) were administered via ear vein IV. Liver samples were fixed and stained with MitoTracker green for mitochondria labeling and ProLong® Gold mounting media with DAPI for nuclear stain. Only limited amounts of T-NPs were found in the liver, large numbers of NT-NPs were localized to liver cells. Scale bar: 25 μm. DIC: differential interference contrast.

FIG. 15 is a number of images. Male C57BL/6 were anesthetized using isofluorane T-QD-NPs: 20 mg/kg with respect to NP was administered via tail vein injection. Distribution of NPs was studied by performing IVIS analyses. The data show the images of all the animals from each group.

FIG. 16 is a number of images. Male C57BL/6 were anesthetized using isofluorane T-QD-NPs: 20 mg/kg with respect to NP was administered tail vein injection. Distribution of NPs in different cell populations in the brain was studied by performing confocal imaging. Immunostaining of tissue sections were performed using antibody treatments against different types of brain cell markers: NeuN for neuronal nuclei, CD-31 for normal endothelial marker, olig2 for oligodendrocytes, and GFAP for astrocytes.

FIG. 17 is a graph. Anti-oxidative properties of T-CoQ10-NPs (1 μM with respect to CoQ10) and comparison with T-Empty-NPs (0.5 mg/mL with respect to total NP) in NSC cells using Seahorse analyzer.

FIG. 18 is a number of graphs. MTT assays on NSCs using CoQ10 and its NPs (top) and prednisone and its NPs (bottom).

FIG. 19 is a number of graphs. Cells were first treated with LPS (100 ng/mL) for 36 h. T-CoQ₁₀-NPs, NTCoQ₁₀-NPs, T-Pred-NPs, NT-Pred-NPs, T-CoQ₁₀-NPs+T-Pred-NPs, NT-CoQ₁₀-NPs+NT-Pred-NPs, free CoQ₁₀, free prednisone, or free prednisone+free CoQ₁₀ (1 μM with respect to CoQ₁₀ or prednisone for all test articles) were added to LPS treated cells. ELISA was performed on the supernatants against IL-4 and IL-12.

FIG. 20 is a number of graphs and images. Dose dependent 14-day toxicity study in piglets. Saline or T-NPs or NTNPs (two different doses, 5 mg/kg and 10 mg/kg with respect to total NP) were administered by intravenous injection and toxicity was followed for 14 days. Representative hematology data from day 7 and day 14 after single intravenous injection of T-NPs, NT-NPs with 5 mg/kg and 10 mg/kg, and saline. Representative images from day 14 post-injection histopathology of brain and liver from treated animals. We were unable to perform hematology analyses on few samples due to clotting.

FIG. 21 is a number of images. (A) Increased ROS levels in saline treated TBI pigs compared to the normal pigs or the TBI pigs treated with a combination of T-CoQ10-NPs+T-Pred-NPs. The TBI piglets treated with NT-CoQ10-NPs+NT-Pred-NPs showed less extent of ROS reduction compared to the ones treated with T-CoQ10-NPs+T-Pred-NPs. ROS was detected by DCF-DA staining in uninjured and injured brain slices. (B) Hematoxylin and eosin staining of brain tissue. Necrosis and hemorrhage was present at the lesion site. Degenerate neutrophils and a small number of macrophages infiltrated the lesion site, and the endothelium in the necrotic area and surrounding neuroparenchyma was very reactive. There was also edema accumulation within the hemisphere ipsilateral to injury. The data show the images for all animals from each group. Images 1-3: Saline treated TBI piglets; images 4-6: T-Pred-NP+T-CoQ₁₀-NP treated TBI piglets; images 7-9: NTPred-NP+NT-CoQ₁₀-NP treated TBI piglets.

The schematic drawings presented herein are not necessarily to scale.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

To effectively cross the blood-brain barrier, nanoparticles desirably are of an appropriate size, have an appropriate charge density and lipophilicity. The nanoparticles can also contain appropriate targeting moieties. The nanoparticles preferably reach an appropriate central nervous system (CNS) target once they cross the blood-brain barrier and preferably deliver their therapeutic payload at or to the target. The therapeutic particles preferably also exert an ameliorative function.

Nanoparticles, as described herein, include, in some embodiments, a hydrophobic core, a hydrophilic layer surrounding the core. The nanoparticles may contain one or more mitochondrial targeting moieties. CNS tissue can be mitochondria-dense tissue, particularly white matter of the brain. Accordingly, mitochondrial targeting moieties may serve as CNS targeting moieties. In addition, mitochondrial targeting may enhance the effects of certain therapeutic agents; particularly those agents that desirably act within mitochondria. For example, because mitochondria are often a source or reactive oxygen species, antioxidants that are targeted to mitochondria may be more effective than antioxidants that are not targeted to the mitochondria. In some embodiments, the nanoparticles described herein include an antioxidant, an anti-inflammatory agent, or both an anti-oxidant and an anti-inflammatory agent. Such agents may be used to treat any suitable disease in a patient in need thereof. In some embodiments, nanoparticles that contain such therapeutic agents are used to treat damages CNS tissue, such as damaged neural tissue. In some embodiments, nanoparticles that contain such therapeutic agents are used to treat traumatic brain injury.

Nanoparticles having a mitochondrial targeting moiety and a photosensitizer may be made in any suitable manner. In embodiments, nanoparticles can be constructed as described in (i) WO 2013/123298, published on Aug. 22, 2012, entitled Nanoparticles for Mitochondrial Trafficking of Agents, and describing information generally as disclosed in Marrache and Dhar (Oct. 2, 2012), Proc. Natl. Acad. Sci. USA, vol. 109 (40), pages 16288-16293; or (ii) WO 2013/033513, published on Mar. 7, 2013, entitled Apoptosis-Targeting Nanoparticles, which claims priority to U.S. Provisional Patent Application No. 61/529,637 filed on Sep. 9, 2012, each of which patent applications and publications are incorporated herein by reference in their respective entireties to the extent that they do not conflict with the present disclosure.

### I. Core

The core of a nanoparticle may be formed from any suitable component or components. Preferably, the core is formed from hydrophobic components such as hydrophobic polymers or hydrophobic portions of polymers. The core may also or alternatively include block copolymers that have hydrophobic portions and hydrophilic portions that may self-assemble in an aqueous environment into particles having the hydrophobic core and a hydrophilic outer surface. In embodiments, the core comprises one or more biodegradable polymer or a polymer having a biodegradable portion.

As used herein, a “hydrophilic” polymer or compound is a polymer or compound that is more soluble in water than in octanol. A “hydrophobic” polymer or compound is a polymer or compound that is more soluble in octanol than in water. In some embodiments, a hydrophilic polymer has a solubility in water of 10 milligrams per liter or greater. In some embodiments, a “hydrophobic” polymer has a solubility in water of 1 milligram per liter or less. The precise chemical structure of a polymer or block is not as important as the degree or hydrophilicity or hydrophobicity because the nanoparticles preferably self-assemble such that hydrophobic components cluster or hydrophilic components cluster under conditions employed for forming the nanoparticles. One of skill in the art of self-assembled nanoparticle synthesis will readily appreciate and understand what polymers are considered hydrophilic and what polymers are considered hydrophobic.

Any suitable synthetic or natural bioabsorbable polymers may be used. Such polymers are recognizable and identifiable by one or ordinary skill in the art. Non-limiting examples of synthetic, biodegradable polymers include: poly(amides) such as poly(amino acids) and poly(peptides); poly(esters) such as poly(lactic acid), poly(glycolic acid), poly(lactic-co-glycolic acid) (PLGA), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or readily identifiable.

In various embodiments, described herein the core comprises PLGA. PLGA is a well-known and well-studied hydrophobic biodegradable polymer used for the delivery and release of therapeutic agents at desired rates. Examples of other hydrophobic polymers include polyacrylics such as polyacrylates, polyacrylonitriles, polymaleic anhydrides, polyacrylates, polymethacrylates, polyamides, polyimide, diene polymers, polyesters, polyethers, fluorocarbon polymers, polyolefins, polystyrenes, polyvinylacetals, polyvinyls, polyvinylchlorides, polyvinylesters, polyvinlyketones, polyvinylpyridines and the like.

Preferably, the at least some of the polymers used to form the core are amphiphilic having hydrophobic portions and hydrophilic portions. The hydrophobic portions can form the core, while the hydrophilic regions may form a layer surrounding the core to help the nanoparticle evade recognition by the immune system and enhance circulation half-life. Examples of amphiphilic polymers include block copolymers having a hydrophobic block and a hydrophilic block. In embodiments, the core is formed from hydrophobic portions of a block copolymer, a hydrophobic polymer, or combinations thereof.

The ratio of hydrophobic polymer to amphiphilic polymer may be varied to vary the size of the nanoparticle. In embodiments, a greater ratio of hydrophobic polymer to amphiphilic polymer results in a nanoparticle having a larger diameter. Any suitable ratio of hydrophobic polymer to amphiphilic polymer may be used. In embodiments, the nanoparticle includes about a 50/50 ratio by weight of amphiphilic polymer to hydrophobic polymer or ratio that includes more amphiphilic polymer than hydrophilic polymer, such as about a 20/80 ratio, about a 30/70 ratio, about a 20/80 ratio, about a 55/45 ratio, about a 60/40 ratio, about a 65/45 ratio, about a 70/30 ratio, about a 75/35 ratio, about a 80/20 ratio, about a 85/15 ratio, about a 90/10 ratio, about a 95/5 ratio, about a 99/1 ratio, or about 100% amphiphilic polymer.

In embodiments, the hydrophobic polymer comprises PLGA, such as PLGA-COOH or PLGA-OH or PLGA-TPP. In embodiments, the amphiphilic polymer comprises PLGA and PEG, such as PLGA-PEG. The amphiphilic polymer may be a dendritic polymer having branched hydrophilic portions. Branched polymers may allow for attachment of more than moiety to terminal ends of the branched hydrophilic polymer tails, as the branched polymers have more than one terminal end.

Nanoparticles having a diameter of about 250 nm or less are generally more effectively targeted to mitochondria than nanoparticles having a diameter of greater than about 250 nm. In embodiments, a nanoparticle effective for mitochondrial targeting has a diameter of about 200 nm or less, 190 nm or less, about 180 nm or less, about 170 nm or less, about 160 nm or less, about 150 nm or less, about 140 nm or less, about 130 nm or less, about 120 nm or less, about 110 nm or less, about 100 nm or less, about 90 nm or less, about 80 nm or less, about 80 nm or less, about 80 nm or less, about 80 nm or less, about 80 nm or less, about 70 nm or less, about 60 nm or less, about 50 nm or less, about 40 nm or less, about 30 nm or less, about 20 nm or less, or about 10 nm or less. In embodiments, a nanoparticle has a diameter of from about 10 nm to about 250 nm, such as from about 20 nm to about 200 nm, from about 50 nm to about 160 nm, from about 60 nm to about 150 nm, from about 70 nm to about 130 nm, from about 80 nm to about 120 nm, from about 80 nm to about 100 nm, or the like. In some embodiments, a nanoparticle has a diameter of from about 30 nanometers to about 150 nanometers.

### II. Hydrophilic Layer Surrounding the Core

The nanoparticles described herein may optionally include a hydrophilic layer surrounding the hydrophilic core. The hydrophilic layer may assist the nanoparticle in evading recognition by the immune system and may enhance circulation half-life of the nanoparticle.

As indicated above, the hydrophilic layer may be formed, in whole or in part, by a hydrophilic portion of an amphiphilic polymer, such as a block co-polymer having a hydrophobic block and a hydrophilic block.

Any suitable hydrophilic polymer or hydrophilic portion of an amphiphilic polymer may form the hydrophilic layer or portion thereof. The hydrophilic polymer or hydrophilic portion of a polymer may be a linear or dendritic polymer. Examples of suitable hydrophilic polymers include polysaccharides, dextran, chitosan, hyaluronic acid, polyethylene glycol, polymethylene oxide, polyethylene oxide, and the like.

In embodiments, a hydrophilic portion of a block copolymer comprises polyethylene glycol (PEG). In embodiments, a block copolymer comprises a hydrophobic portion comprising PLGA and a hydrophilic portion comprising PEG.

A hydrophilic polymer or hydrophilic portion of a polymer may contain moieties that are charged under physiological conditions, which may be approximated by a buffered saline solution, such as a phosphate or citrate buffered saline solution, at a pH of about 7.4, or the like. Such moieties may contribute to the charge density or zeta potential of the nanoparticle. Zeta potential is a term for electrokinetic potential in colloidal systems. While zeta potential is not directly measurable, it can be experimentally determined using electrophoretic mobility, dynamic electrophoretic mobility, or the like.

It has been found that zeta potential may play an important role in the ability of nanoparticles to accumulate in mitochondria, with higher zeta potentials generally resulting in increased accumulation in the mitochondria. In embodiments, the nanoparticles have a zeta potential, as measured by dynamic light scattering, of about 0 mV or greater. For example, a nanoparticle may have a zeta potential of about 1 mV or greater, of about 5 mV or greater, of about 7 mV or greater, or about 10 mV or greater, or about 15 mV or greater, of about 20 mV or greater, about 25 mV or greater, about 30 mV or greater, about 34 mV or greater, about 35 mV or greater, or the like. In embodiments, a nanoparticle has a zeta potential of from about 0 mV to about 100 mV, such as from about 1 mV to 50 mV, from about 2 mV to about 40 mV, from about 7 mV to about 35 mV, or the like.

Any suitable moiety that may be charged under physiological conditions may be a part of or attached to a hydrophilic polymer or hydrophilic portion of a polymer. In embodiments, the moiety is present at a terminal end of the polymer or hydrophilic portion of the polymer. Of course, the moiety may be directly or indirectly bound to the polymer backbone at a location other than at a terminal end. Due to the substantial negative electrochemical potential maintained across the inner mitochondrial membrane, cations, particularly if delocalized, are effective at crossing the hydrophobic membranes and accumulating in the mitochondrial matrix. Cationic moieties that are known to facilitate mitochondrial targeting are discussed in more detail below. However, cationic moieties that are not particularly effective for selective mitochondrial targeting may be included in nanoparticles or be bound to hydrophilic polymers or portions of polymers. In embodiments, anionic moieties may form a part of or be attached to the hydrophilic polymer or portion of a polymer. The anionic moieties or polymers containing the anionic moieties may be included in nanoparticles to tune the zeta potential, as desired. In embodiments, a hydrophilic polymer or portion of a polymer includes a hydroxyl group that can result in an oxygen anion when placed in a physiological aqueous environment. In embodiments, the polymer comprises PEG-OH where the OH serves as the charged moiety under physiological conditions.

### III. Mitochondria Targeting Moieties

The nanoparticles described herein include one or more moieties that target the nanoparticles to mitochondria. As used herein, “targeting” a nanoparticle to mitochondria means that the nanoparticle accumulates in mitochondria relative to other organelles or cytoplasm at a greater concentration than substantially similar non-targeted nanoparticle. A substantially similar non-target nanoparticle includes the same components in substantially the same relative concentration (e.g., within about 5%) as the targeted nanoparticle, but lacks a targeting moiety.

The mitochondrial targeting moieties may be tethered to the core in any suitable manner, such as binding to a molecule that forms part of the core or to a molecule that is bound to the core. In embodiments, a targeting moiety is bound to a hydrophilic polymer that is bound to a hydrophobic polymer that forms part of the core. In embodiments, a targeting moiety is bound to a hydrophilic portion of a block copolymer having a hydrophobic block that forms part of the core.

The targeting moieties may be bound to any suitable portion of a polymer. In embodiments, the targeting moieties are attached to a terminal end of a polymer. In embodiments, the targeting moieties are bound to the backbone of the polymer, or a molecule attached to the backbone, at a location other than a terminal end of the polymer. More than one targeting moiety may be bound to a given polymer. In embodiments, the polymer is a dendritic polymer having multiple terminal ends and the targeting moieties may be bound to more than one of terminal ends.

The polymers, or portions thereof, to which the targeting moieties are bound may contain, or be modified to contain, appropriate functional groups, such as —OH, —COOH, —NH₂, —SH, —N₃, —Br, —Cl, —I, or the like, for reaction with and binding to the targeting moieties that have, or are modified to have, suitable functional groups.

Examples of targeting moieties tethered to polymers presented throughout this disclosure for purpose of illustrating the types of reactions and tethering that may occur. However, one of skill in the art will understand that tethering of targeting moieties to polymers may be carried out according to any of a number of known chemical reaction processes.

Targeting moieties may be present in the nanoparticles at any suitable concentration. In embodiments, the concentration may readily be varied based on initial in vitro analysis to optimize prior to in vivo study or use. In embodiments, the targeting moieties will have surface coverage of from about 5% to about 100%.

Any suitable moiety for facilitating accumulation of the nanoparticle within the mitochondrial matrix may be employed. Due to the substantial negative electrochemical potential maintained across the inner mitochondrial membrane, delocalized lipophilic cations are effective at crossing the hydrophobic membranes and accumulating in the mitochondrial matrix. Triphenyl phosophonium (TPP) containing compounds can accumulate greater than 1000 fold within the mitochondrial matrix. Any suitable TPP-containing compound may be used as a mitochondrial matrix targeting moiety. Representative examples of TPP-based moieties may have structures indicated below in Formula I, Formula II or Formula III:

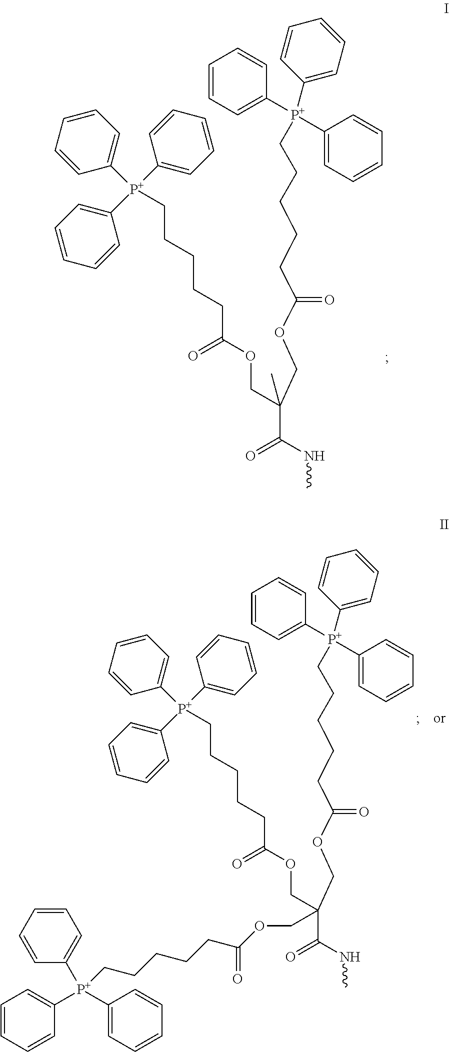

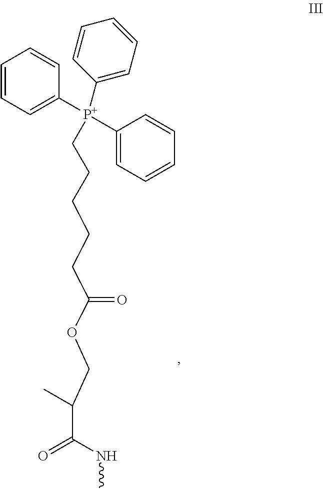

where the amine (as depicted) may be conjugated to a polymer or other component for incorporation into the nanoparticle.

In embodiments, the delocalized lipophilic cation for targeting the mitochondrial matrix is a rhodamine cation, such as Rhodamine 123 having Formula IV as depicted below:

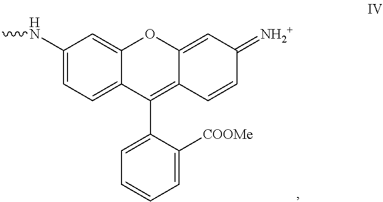

where the secondary amine (as depicted) may be conjugated to a polymer, lipid, or the like for incorporation into the nanoparticle.

Of course, non-cationic compounds may serve to target and accumulate in the mitochondrial matrix. By way of example, Szeto-Shiller peptide may serve to target and accumulate a nanoparticle in the mitochondrial matrix. Any suitable Szetto-Shiller peptide may be employed as a mitochondrial matrix targeting moiety. Non-limiting examples of suitable Szeto-Shiller peptides include SS-02 and SS-31, having Formula V and Formula VI, respectively, as depicted below:

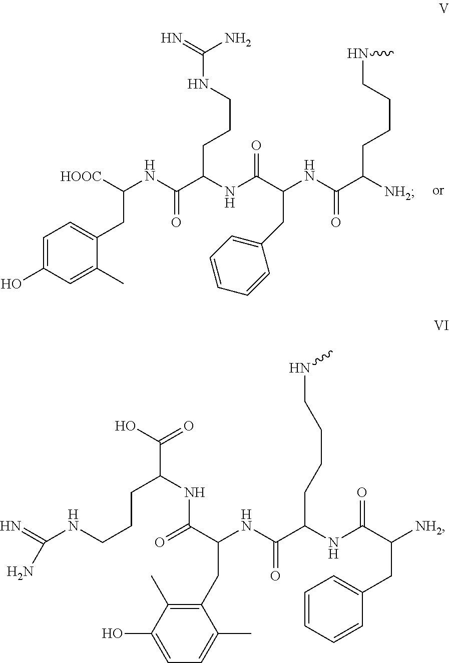

where the secondary amine (as depicted) may be conjugated to a polymer, lipid, or the like for incorporation into the nanoparticle.

For purposes of example, a reaction scheme for synthesis of PLGA-PEG-TPP is shown below in Scheme I. It will be understood that other schemes may be employed to synthesize PLGA-PEG-TPP and that similar reaction schemes may be employed to tether other mitochondrial targeting moieties to PLGA-PEG or to tether moieties to other polymer or components of a nanoparticle.

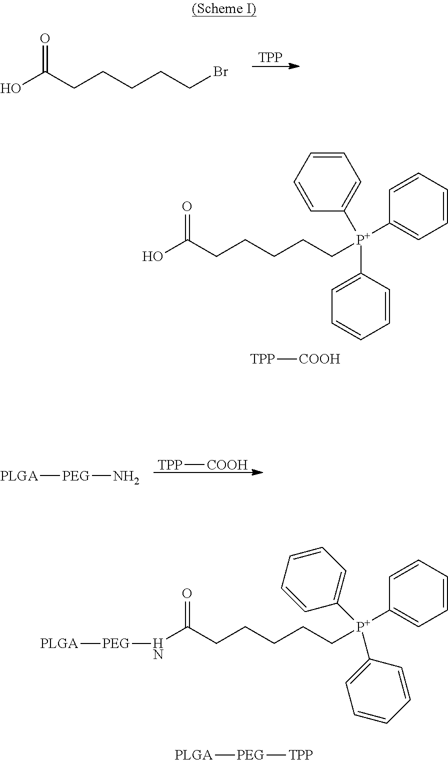

Preferably, a targeting moiety is attached to a hydrophilic polymer or hydrophilic portion of a polymer so that the targeting moiety will extend from the core of the nanoparticle to facilitate the effect of the targeting moiety.

It will be understood that the mitochondrial targeting moiety may alter the zeta potential of a nanoparticle. Accordingly, the zeta potential of a nanoparticle may be tuned by adjusting the amount of targeting moiety included in the nanoparticle. The zeta potential may also be adjusted by including other charged moieties, such as charged moieties of, or attached to, hydrophilic polymers or hydrophilic portions of polymers.

In embodiments, charged moieties are provided only by, or substantially by, mitochondrial targeting moieties. In embodiments, about 95% or more of the charged moieties are provided by mitochondrial targeting moieties. In embodiments, about 90% or more of the charged moieties are provided by mitochondrial targeting moieties. In embodiments, about 85% or more of the charged moieties are provided by mitochondrial targeting moieties. In embodiments, about 80% or more of the charged moieties are provided by mitochondrial targeting moieties. In embodiments, about 75% or more of the charged moieties are provided by mitochondrial targeting moieties. In embodiments, about 70% or more of the charged moieties are provided by mitochondrial targeting moieties. In embodiments, about 65% or more of the charged moieties are provided by mitochondrial targeting moieties. In embodiments, about 60% or more of the charged moieties are provided by mitochondrial targeting moieties. In embodiments, about 55% or more of the charged moieties are provided by mitochondrial targeting moieties. In embodiments, about 50% or more of the charged moieties are provided by mitochondrial targeting moieties. Of course, the mitochondrial targeting moieties may provide any suitable amount or percentage of the charged moieties.

In embodiments, the nanoparticles are formed by blending a polymer to which a mitochondrial targeting moiety is attached with a polymer to which a charged moiety other than a mitochondrial targeting moiety is attached.

### IV. Antioxidant

A nanoparticle, as described herein, may include any one or more antioxidants. Preferably, the one or more antioxidants react with reactive oxygen species. A reactive oxygen species is a chemically reactive molecule containing oxygen. Examples of reactive oxygen species are molecules that include oxygen ions, oxygen radicals, peroxides, and the like. The one or more antioxidant may be embedded in, or contained within, the core of the nanoparticle. Preferably, the antioxidant is released from the core at a desired rate. If the core is formed from a polymer (such as PLGA) or combination of polymers having known release rates, the release rate can be readily controlled.

In embodiments, an antioxidant or precursor thereof is conjugated to a polymer, or other component of a nanoparticle, in a manner described above with regard to targeting moieties. The antioxidant may be conjugated via a cleavable linker so that the antioxidant may be released when the nanoparticle reaches the target location, such as mitochondria.

The antioxidant may be present in the nanoparticle at any suitable concentration. For example, an antioxidant may be present in the nanoparticle at a concentration from about 0.0001% to about 40% by weight of the nanoparticle.

Any suitable antioxidant may be used. Examples of antioxidants include glutathione, vitamin C, vitamin A, vitamin E, calalase, superoxise dismutate, a peroxidase, coenzyme Q₁₀ (coQ₁₀), and the like.

In embodiments, the antioxidant is CoQ₁₀. CoQ₁₀ is present in most cells, primarily in the mitochondria, and is a component of the electron transport chain. CoQ₁₀ can exist in a fully oxidized form (ubiquinone), a partially oxidized form (ubisemiquinone) and a fully reduced form (ubiquinol). Preferably, the CoQ₁₀ is ubisemiquinone or ubiquinol.

### V. Anti-Inflammatory Agents

A nanoparticle, as described herein, may include any one or more anti-inflammatory agent. The one or more anti-inflammatory agent may be embedded in, or contained within, the core of the nanoparticle. Preferably, the anti-inflammatory agent is released from the core at a desired rate. If the core is formed from a polymer (such as PLGA) or combination of polymers having known release rates, the release rate can be readily controlled.

In embodiments, an anti-inflammatory agent or precursor thereof is conjugated to a polymer, or other component of a nanoparticle, in a manner described above with regard to targeting moieties. The anti-inflammatory agent may be conjugated via a cleavable linker so that the anti-inflammatory agent may be released when the nanoparticle reaches the target location, such as mitochondria.

The anti-inflammatory agent may be present in the nanoparticle at any suitable concentration. For example, an anti-inflammatory agent may be present in the nanoparticle at a concentration from about 0.0001% to about 40% by weight of the nanoparticle.

Any suitable anti-inflammatory agent may be used. Examples of anti-inflammatory agents include steroidal anti-inflammatory agents, nonsteroidal anti-inflammatory agents, and the like. In some embodiments, anti-inflammatory agents include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium. triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

In embodiments, the anti-inflammatory agent is prednisone.

### VI. Imaging Agents

A nanoparticle, as described herein, may include any one or more imaging agent. The one or more imaging agent may be embedded in, or contained within, the core of the nanoparticle, or attached to the nanoparticle. Any suitable imaging agent can be used. In some embodiments, the imaging agent is one or more of a fluorphore, a magnetic agent or a radioactive agent. Examples of imaging agents include ⁶⁴Cu diacetyl-bis(N⁴-methylthiosemicarbazone), ¹⁸F-fluorodeoxyglucose, 3′-deoxy-3′-[¹⁸F]fluorothymidine, gallium, technetium-99m, thallium, barium, gastrograin, iodine constrast agents, iron oxide, and quantum dots.

Preferably, the imaging agent is therapeutically or diagnostically relevant. Examples of therapeutically or diagnostically relevant imaging agents include imaging agents attached to molecules that target the imaging agent to a cell or molecule associated with a particular disease. By way of example, a target for a cancer cell may be an oncogene, a mutant tumor suppressor, or the like. Examples of targeting molecules include antibodies, polynucleotides, receptor agonist or antagonist, and the like.

### VI. Synthesis of Nanoparticle

Nanoparticles, as described herein, may be synthesized or assembled via any suitable process. Preferably, the nanoparticles are assembled in a single step to minimize process variation. A single step process may include nanoprecipitation and self-assembly.

In general, the nanoparticles may be synthesized or assembled by dissolving or suspending hydrophobic components in an organic solvent, preferably a solvent that is miscible in an aqueous solvent used for precipitation. In embodiments, acetonitrile is used as the organic solvent, but any suitable solvent (such as DMF, DMSO, acetone, or the like) may be used. Hydrophilic components are dissolved in a suitable aqueous solvent, such as water, 4 wt-% ethanol, or the like. The organic phase solution may be added drop wise to the aqueous phase solution to nanoprecipitate the hydrophobic components and allow self-assembly of the nanoparticle in the aqueous solvent.

A process for determining appropriate conditions for forming the nanoparticles may be as follows. Briefly, functionalized polymers and other components, if included or as appropriate, may be co-dissolved in organic solvent mixtures. This solution may be added drop wise into hot (e.g, 65° C.) aqueous solvent (e.g, water, 4 wt-% ethanol, etc.), whereupon the solvents will evaporate, producing nanoparticles with a hydrophobic core surrounded by a hydrophilic polymer component, such as PEG. Once a set of conditions where a high (e.g., >75%) level of targeting moiety surface loading has been achieved, contrast agents or therapeutic agents may be included in the nanoprecipitation and self-assembly of the nanoparticles.

If results are not desirably reproducible by manual mixing, microfluidic channels may be used.

Nanoparticles may be characterized for their size, charge, stability, loading, drug release kinetics, surface morphology, and stability using well-known or published methods.

Nanoparticle properties may be controlled by (a) controlling the composition of the polymer solution, and (b) controlling mixing conditions such as mixing time, temperature, and ratio of water to organic solvent. The likelihood of variation in nanoparticle properties increases with the number of processing steps required for synthesis.

The size of the nanoparticle produced can be varied by altering the ratio of hydrophobic core components to amphiphilic shell components. Nanoparticle size can also be controlled by changing the polymer length, by changing the mixing time, and by adjusting the ratio of organic to the phase. Prior experience with nanoparticles from PLGA-b-PEG of different lengths suggests that nanoparticle size will increase from a minimum of about 20 nm for short polymers (e.g. PLGA₃₀₀₀-PEG₇₅₀) to a maximum of about 150 nm for long polymers (e.g. PLGA_{100,000}-PEG_{10,000}). Thus, molecular weight of the polymer will serve to adjust the size.

Nanoparticle surface charge can be controlled by mixing polymers with appropriately charged end groups. Additionally, the composition and surface chemistry can be controlled by mixing polymers with different hydrophilic polymer lengths, branched hydrophilic polymers, or by adding hydrophobic polymers.

Once formed, the nanoparticles may be collected and washed via centrifugation, centrifugal ultrafiltration, or the like. If aggregation occurs, nanoparticles can be purified by dialysis, can be purified by longer centrifugation at slower speeds, can be purified with the use surfactant, or the like.

Once collected, any remaining solvent may be removed and the particles may be dried, which should aid in minimizing any premature breakdown or release of components. The nanoparticles may be freeze dried with the use of bulking agents such as mannitol, or otherwise prepared for storage prior to use.

It will be understood that therapeutic agents may be placed in the organic phase or aqueous phase according to their solubility.

Nanoparticles described herein may include any other suitable components, such as phospholipids or cholesterol components, generally know or understood in the art as being suitable for inclusion in nanoparticles. WO 2013/033513, for example, describes a number of additional components that may be included in nanoparticles.

### VII. Use and Testing

In general, a nanoparticle as described herein may be administered systemically to a patient in need thereof. For purposes of the present disclosure, “systemic administration” means administration outside of the CNS. Systemic administration includes oral, IV, IP, and the like. In some embodiments, the nanoparticles are administered to a patient suffering from or at risk of damaged CNS tissue, such as damaged neural tissue. In some embodiments, the nanoparticles are administered to a patient suffering from traumatic brain injury.

The performance and characteristics of nanoparticles produced herein may be tested or studied in any suitable manner. By way of example, therapeutic efficacy can be evaluated using cell-based assays. Toxicity, bio-distribution, pharmacokinetics, and efficacy studies can be tested in cells or rodents or other mammals. Zebrafish or other animal models may be employed for combined imaging and therapy studies. Rodents, rabbits, pigs, or the like may be used to evaluate diagnostic or therapeutic potential of nanoparticles. Some additional details of studies that may be performed to evaluate the performance or characteristics of the nanoparticles, which may be used for purposes of optimizing the properties of the nanoparticles are described below. However, one of skill in the art will understand that other assays and procedures may be readily performed.

Uptake and binding characteristics of nanoparticles containing a contrast agent may be evaluated in any suitable cell line, such as RAW 264.7, J774, jurkat, and HUVEGs cells. The immunomodulatory role of nanoparticles may be assayed by determining the release of cytokines when these cells are exposed to varying concentrations of nanoparticles. Complement activation may be studied to identify which pathways are triggered using columns to isolate opsonized nanoparticles; e.g. as described in Salvador-Morales C, Zhang L, Langer R, Farokhzad O C, Immunocompatibility properties of lipid-polymer hybrid nanoparticles with heterogeneous surface functional groups, Biomaterials 30: 2231-2240, (2009). Fluorescence measurements may be carried out using a plate reader, FACS, or the like. Because nanoparticle size is an important factor that determines biodistribution, Nanoparticles may be binned into various sizes (e.g., 20-40, 40-60, 60-80, 80-100, 100-150, and 150-300 nm) and tested according to size.

Any cell type appropriate for an antioxidant or anti-inflammatory agent employed in a nanoparticle may be used to evaluate therapeutic efficacy or proper targeting. Assays appropriate for the therapeutic or pharmacologic outcome may be employed, as are generally understood or known in the art.

Biodistribution (bioD) and pharmacokinetic (PK) studies may be carried out in rats, pigs or other suitable mammals. For example, Sprague Dawley rats may be dosed with QD-labeled, mitochondria-targeting nanoparticles or similar nanoparticles without the targeting groups, through a lateral tail vein injection for PK and bioD analysis. The bioD may be followed initially by fluorescence imaging for 1-24 h after injection. Animals may be sacrificed; and brain, heart, intestine, liver, spleen, kidney, muscle, bone, lung, lymph nodes, gut, and skin may be excised, weighed, homogenized, and Cd from QD may be quantified using ICP-MS. Tissue concentration may be expressed as % of injected dose per gram of tissue (% ID/g). Blood half-life may be calculated from blood Cd concentrations at various time points

Therapeutic dosages of nanoparticles effective for human use can be estimated from animal studies according to well-known techniques, such as surface area or weight based scaling.

The nanoparticles described herein have been shown to accumulate in the brain. Accordingly, the nanoparticles described herein can be used to treat or diagnose brain related diseases. Examples of brain related diseases include brain injury, stroke, traumatic brain injury, brain cancer, infection, Parkinson's disease, Huntington's disease, Alzheimer's disease, and the like. A targeting molecule can be associated with, or attached to, the nanoparticle, therapeutic agent, or imaging agent to target the nanoparticle, therapeutic agent or imaging agent to a diseased cell.

### IX. Definitions

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like.

As used herein, “disease” means a condition of a living being or one or more of its parts that impairs normal functioning. As used herein, the term disease encompasses terms such disease, disorder, condition, dysfunction and the like.

As used herein, “treat” or the like means to cure, prevent, or ameliorate one or more symptom of a disease.

As used herein, “bind,” “bound,” or the like means that chemical entities are joined by any suitable type of bond, such as a covalent bond, an ionic bond, a hydrogen bond, van der walls forces, or the like. “Bind,” “bound,” and the like are used interchangeable herein with “attach,” “attached,” and the like.

As used herein, a molecule or moiety “attached” to a core of a nanoparticle may be embedded in the core, contained within the core, attached to a molecule that forms at least a portion of the core, attached to a molecule attached to the core, or directly attached to the core.

As used herein, a “derivative” of a compound is a compound structurally similar to the compound of which it is a derivative. Many derivatives are functional derivatives. That is, the derivatives generally a desired function similar to the compound to which it is a derivative. By way of example, triphenyl phosophonium (TPP) is described herein as a mitochondrial targeting moiety because it can accumulate, or cause a compound or complex (such as a nanoparticle) to which it is bound to accumulate, in the mitochondrial matrix. Accordingly, a functional derivative of TPP is a derivative of TPP that may accumulate, or cause a compound or complex to which it is bound to accumulate, in the mitochondrial matrix in a similar concentration as TPP (e.g., within about a 100 fold concentration range, such as within about a 10 fold concentration range).

In the following, non-limiting examples are presented, which describe various embodiments of representative nanoparticles, methods for producing the nanoparticles, and methods for using the nanoparticles.

### Examples

Traumatic brain injury (TBI) is one of the leading causes of death and long-term disability in both civilian life and the battlefield worldwide. Beyond the primary injury caused by the initial insult, a cascaded of events rapidly occur including the production of free radicals and heightened immune response which result in considerable secondary injury to brain tissue. Significant efforts made by the medical and research communities to develop neuroprotective therapeutics that will limit secondary injury led to numerous clinical trials. Despite years of research and advancements, there are no neuroprotective treatment options that exist with improved neurological outcomes. One potential option to improve efficacy is the use of a nanoparticle delivery system that is of optimized size, charge, lipophilicity, and targeting properties to cross the blood-brain barrier (BBB) and can reach specific intracellular targets to deliver neuroprotectant payloads.

In this study, we examined for the first time the therapeutic potential of a highly lipophilic BBB penetrating biodegradable mitochondria targeted nanoparticle (NP) containing an antioxidant and an anti-inflammatory cocktail therapy in a piglet model of TBI. The targeted NP was found to distribute in the lipophilic white matter of normal pig brain. Evaluation of the targeted NP in a piglet model of TBI demonstrated favorable pharmacokinetics and unique distribution in the injured brain. The targeted NP system was further engineered to carry a mitochondria-acting antioxidant coenzyme Q10 and an anti-inflammatory agent prednisone. Therapeutic potential of the targeted NP containing cocktail therapy in neuronal stem cells demonstrated unique abilities to reduce oxidative stress and anti-inflammatory properties. This technology has the potential to provide therapeutic effects against the cascade of events which rapidly occur after TBI including the production of free radicals and heightened immune response which result in considerable secondary injury to brain tissue.

Traumatic brain injury (TBI) is a “silent epidemic” as one of the leading causes of death and long-term disability among persons in the United States. More than 1.7 million individuals suffer a TBI annually with approximately 50,000 patient deaths and 80,500 patients with long-term disabilities. The life quality of TBI survivors is often significantly reduced with victims suffering from learning and memory problems, challenges with language, decision making, problem solving, motor function and afflicted with chronic fatigue, depression and emotional instability. The catastrophic nature of TBI is heightened by the fact that children less than five years old are the demographic that suffer the highest incidence of TBI-related hospitalizations and deaths. The sequel of TBI is initiated by a primary injury to the brain that is rapidly followed by a cascade of secondary events including oxidative and inflammatory insults that further exacerbate tissue loss and damage and ultimately brain function. It is this secondary injury cascade that has become a prime target for therapeutic intervention.

The TBI secondary injury cascade has proven to be a complex series of mechanisms and events that lead to the destruction of brain tissue at the cellular level with two major components being the formation of free radicals and immune response. After initial trauma to the brain, a series of catabolic processes lead to an increase in intracellular concentration of free radicals including reactive oxygen species (ROS). Increased ROS production leads to peroxidation of cellular structures, cleavage of DNA, and disruption of the mitochondrial electron transport chain (ETC). Simultaneously, a mounting immune response further heightens secondary injury. Microglia and infiltrating macrophages secrete inflammatory cytokines and chemokines such as tumor necrosis factor-alpha (TNF-α) and interleukin-8 (IL-8) that lead to increased levels of cellular apoptosis. TNF-α signals through its cognate receptors TNFR1 and TNFR2 to induce cell death via caspase-8-mediated apoptosis. The cell ultimately fragments into apoptotic cell bodies that are engulfed by neighboring cells. The expression of these inflammatory cytokines and chemokines are shown to be significantly upregulated in both human brains and serum samples post-TBI and are used as biomarkers to assess the extent of head trauma.

Neuroprotectants that inhibit secondary injury cascades have led to a number of phase II and III clinical trials. However, despite promising data in preclinical rodent animal models and early phase randomized and double blinded trials, no neuroprotective treatment option exists that leads to improved neurological outcomes. One potential option to improve efficacy is to improve therapeutic delivery through approaches that are better able to cross the blood-brain barrier (BBB), better target cells, and cellular compartments of interest. Only a handful of small lipophilic molecules, including therapeutic antioxidants and anti-inflammatory agents that could ameliorate secondary injury, can freely diffuse across the lipid membranes of the BBB. However, increasing the lipophilicity of a small molecule-based drug to enhance BBB penetration can have undesirable effects, which include reduced overall solubility, and bioavailability, increased plasma protein interactions, and increased uptake by the cells of the mononuclear phagocyte system (MPS). Delivery of neuroprotectants to the brain can potentially be achieved by using biodegradable nanoparticles (NPs) engineered to achieve three milestones: (1) optimize size charge, lipophilicity, and targeting properties to cross the BBB into the brain, (2) reach specific intracellular targets and demonstrate controlled release of the payload at the target, and (3) performing its ameliorative function there. This would enable effective and robust delivery of neuroprotectants to injured cells and stem cellular degradation and tissue damage. High mitochondrial density in cerebral endothelial cells than in peripheral endothelia provide a strategy to target brain by constructing a highly lipophilic mitochondria targeted NP system with suitable size and charge.

Most studies have focused on rodent models for the development of neuroprotectants, but in part this has likely contributed to the limited translatability of therapeutic findings developed in the rodent to human patients. To enhance the therapeutic potential, efforts have been made to work in models more similar to humans such as the pig. Compared to the rodent, the piglet brain has greater anatomical and physiological similarities to humans with comparable gray-white matter composition, brain size, and both having gyrencephalic brains unlike rodents. This suggests that the pig TBI model may be more advantageous for neural injury as outcomes are likely to be more predictive of what would occur in a human brain. In addition, we examined the localization of highly lipophilic BBB penetrating biodegradable mitochondria targeted nanoparticle (NP) containing an imaging agent in brain.

In this study, we examined the therapeutic potential of a highly lipophilic BBB penetrating biodegradable mitochondria targeted nanoparticle (NP) containing an antioxidant and an anti-inflammatory cocktail therapy in a piglet model of TBI.

### Results and Discussion

### Mitochondria Targeted Lipophilic NPs for TBI.

One consideration in developing a NP system in delivering a combination of an antioxidant and an anti-inflammatory agent is the ability of the NP to cross the blood-brain barrier (BBB). Furthermore, the intracellular location of oxidative stress is the mitochondria of cells and the target organelle of most antioxidants is mitochondria of cells. Moreover, the combination of roles of in pro-inflammatory signaling and abilities of proinflammatory mediators to alter mitochondrial function increase mitochondrial oxidative stress, promoting a vicious inflammatory cycle. Thus, strategies aimed at controlling excessive oxidative and inflammatory stress within mitochondria may represent both preventive and therapeutic interventions in inflammation. Thus the NP system for delivery of antioxidants, anti-inflammatory agents, or antioxidants and anti-inflammatory agents in brain tissue preferably has both BBB and mitochondria targeting properties.

The BBB is formed by endothelial cells in the brain lining the cerebral vasculature that form tight junctions that are 50-100 times tighter than peripheral microvessels. In addition, astrocytic endfeet form “rosette”-like structures around the brain capillary surface and communicate to the endothelial cells to regulate blood flow and nutrient supply. The BBB is an important mechanism in protecting the brain from fluctuations in plasma composition and in maintaining homeostasis in the brain microenvironment. These tight junctions restrict hydrophilic solutes from diffusing out of the brain capillaries, so penetration of the BBB is effectively confined to transcellular mechanisms. Thus, pass from blood to brain of circulating NPs may only happen by transcellular mechanisms, which require a highly lipophilic NP with suitable size and charge. Brain endothelial cell surface and basement membrane components bearing highly anionic charges from sulfated proteoglycans are different from non-brain endothelium and would allow the adsorptive-mediated transcytosis of cationic NPs. Thus the small size and highly lipophilic surface of NPs can help their distribution in the brain. Furthermore, high mitochondrial density in cerebral endothelial cells than in peripheral endothelia provide a strategy to target brain by constructing a highly lipophilic mitochondria targeted NP system with suitable size and charge.

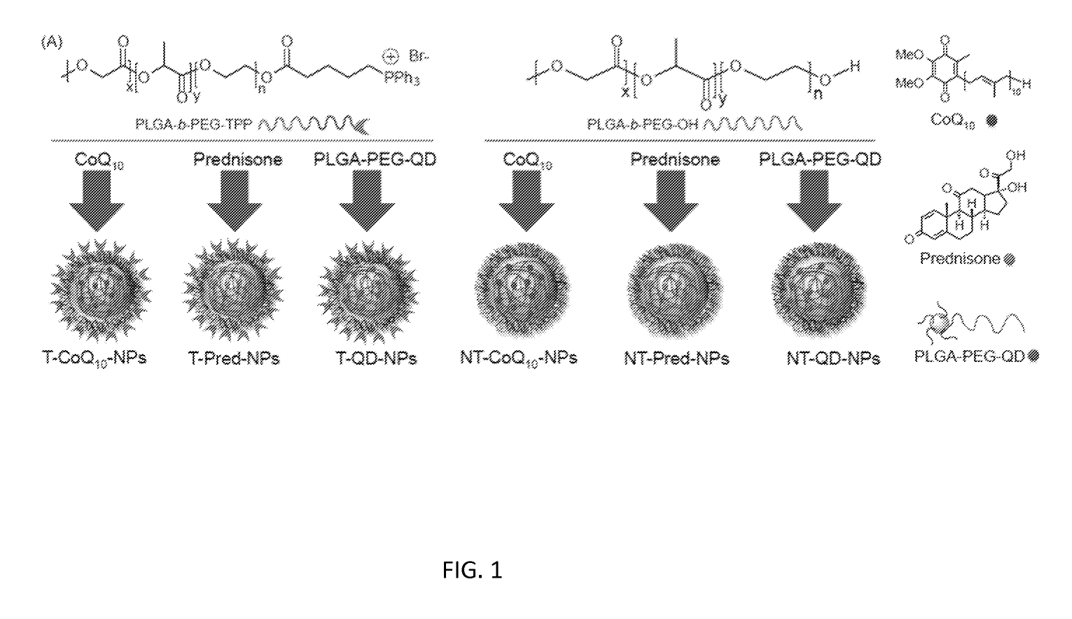

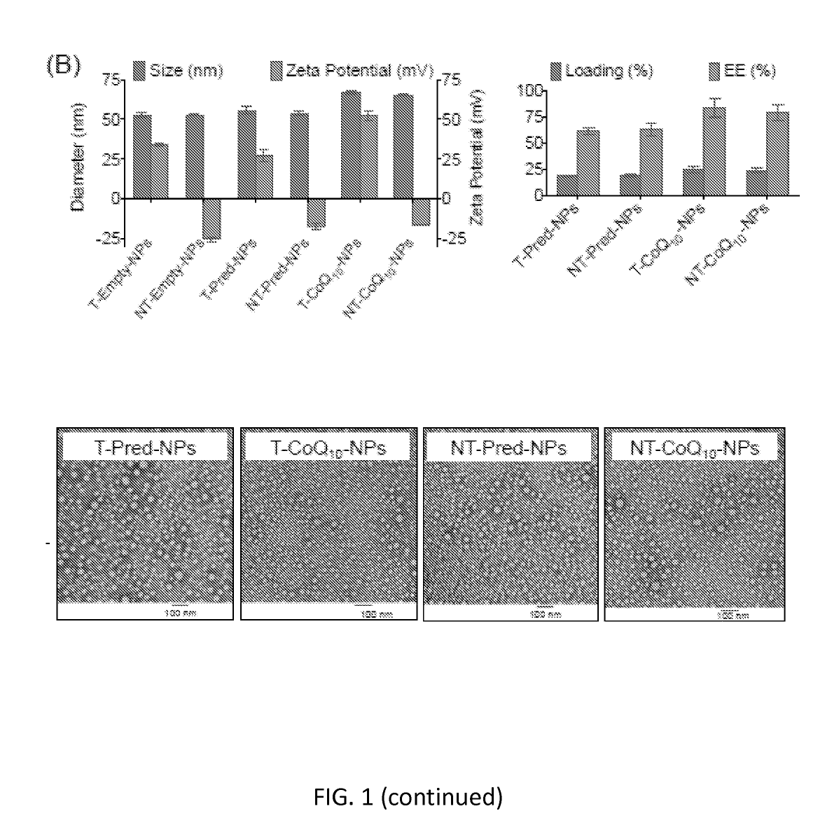

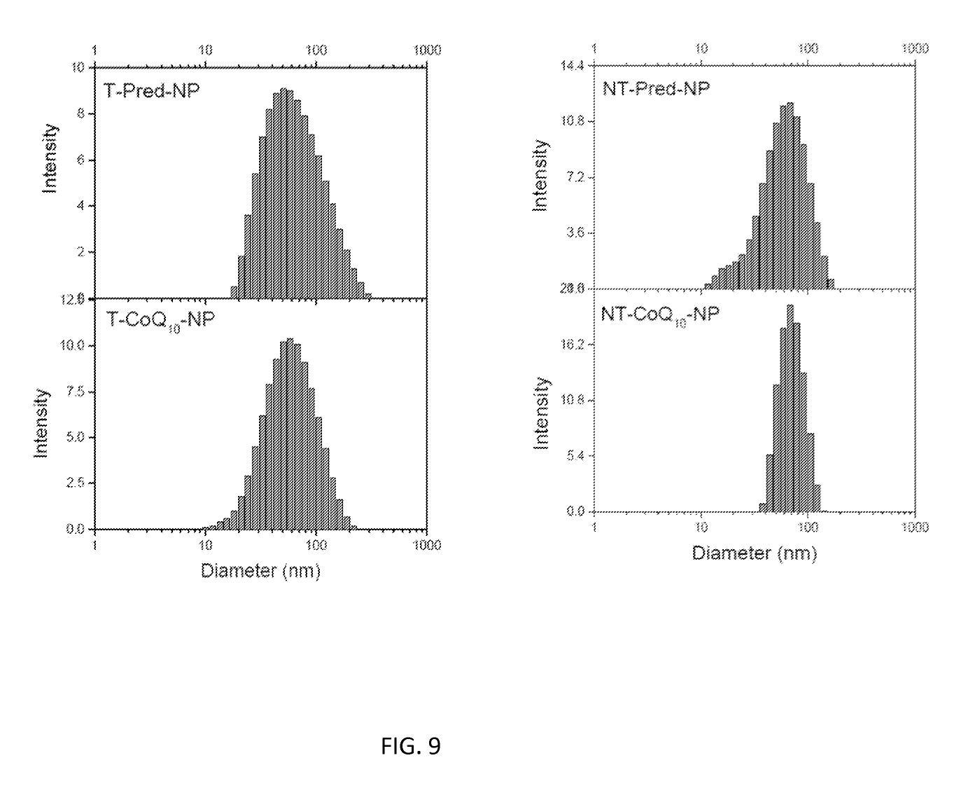

We recently developed a biocompatible polymeric NP based on biodegradable poly(lactic-co-glycolic acid) (PLGA)-block (b)-polyethyleneglycol (PEG) functionalized with a terminal triphenylphosphonium (TPP) cation which has efficient with brainpenetrating properties and remarkable activity to target mitochondria of cells due to its high lipophilic properties, presence of delocalized positive charge, and appropriate size range. The TPP cation in PLGA-b-PEG-TPP polymer takes advantage of the substantial negative ΔΨₘ across the inner mitochondrial membrane (IMM) to efficiently accumulate inside the matrix. In rodent model, an optimized formulation of targeted NPs (T-NPs) derived from PLGA-b-PEG-TPP polymer was found to accumulate in the brain efficiently. In a rat bioD model, T-NPs were delivered to the brain in the case of intravenous administration. This formulation was also found to accumulate in the mitochondria matrix. It is reported in the literature that low molecular weight TPP cation containing small molecules are taken up into the brain, however the extent of uptake is less than into other tissues and the extent of brain distribution correlates with the hydrophobicity of the compound. We believe that incorporation of −TPP cation on NP surface creates a hydrophobic delocalized cationic surface which play significant roles in the brain accumulation of these NPs. We therefore, used this particular NP formulation to encapsulate a mitochondria-acting antioxidant coenzyme Q₁₀ (CoQ₁₀) to develop T-CoQ₁₀-NPs (FIG. 1A). The non-targeted polymer PLGA-b-PEG-OH devoid of a mitochondria targeting lipophilic TPP moiety was used to generate control NP formulation NT-CoQ10-NPs. For anti-inflammatory effects, prednisone, a synthetic corticosteroid drug was used and T-Prednisone-NPs and NT-Prednisone-NPs were formulated (FIG. 1B, FIG. 9, Table 2). The targeted (T) and non-targeted (NT) NPs were constructed by incorporating a polymer conjugated quantum dot (QD), PLGA-PEG-QD to result T-QD-NPs and NT-QD-NPs for biodistribution (bioD) and pharmacokinetic (PK) profile measurements (FIG. 1A, Table 3). The targeted polymer PLGA-b-PEG-TPP and the non-targeted control PLGA-b-PEG-OH were synthesized and characterized following methods previously described by Marrache and Dhar, Proc. Natl. Acac. Sci. USA 2012, 109, 16288-16293. The NPs were characterized by dynamic light scattering (DLS) to give the size, polydispersity index (PDI), and zeta potential of each preparation (FIG. 1B, FIG. S1, Table S1). The small size and high positive zeta potential of the T-NPs indicated that these NPs will be suitable for BBB crossing and mitochondrial uptake properties. Loading efficiencies of CoQ10 and prednisone at various added weight-percentage values of these drugs to polymer indicated that both CoQ10 and prednisone can be entrapped in these NPs with a very high loading and encapsulation efficiency (EE) (FIG. 1B, FIG. 9, Table 2). Morphology of these NPs was investigated using transmission electron microscopy (TEM) (FIG. 1B).

### Distribution of T-NPS in the Lipophilic White Matter of Brain.

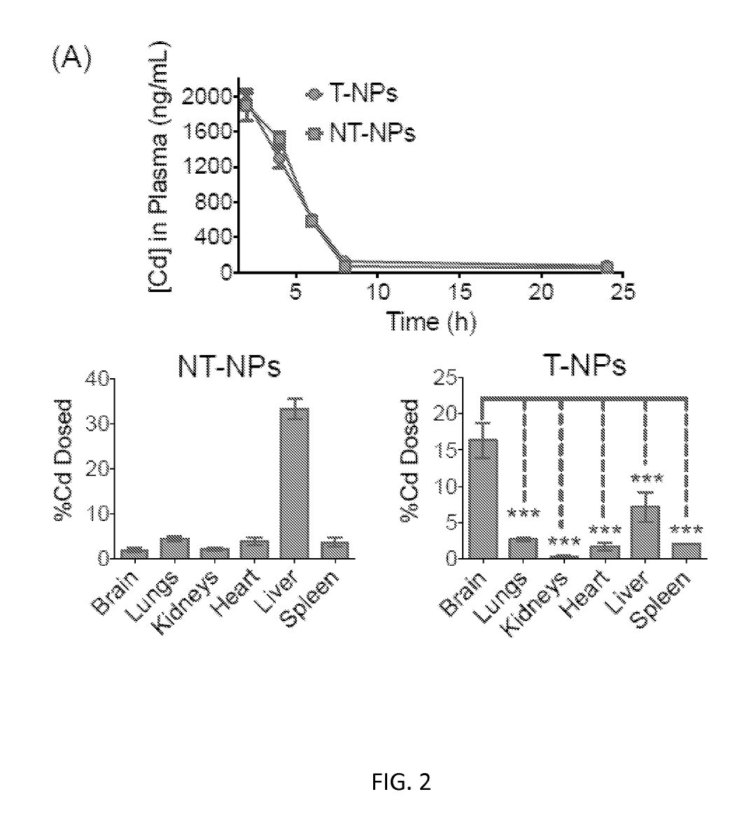

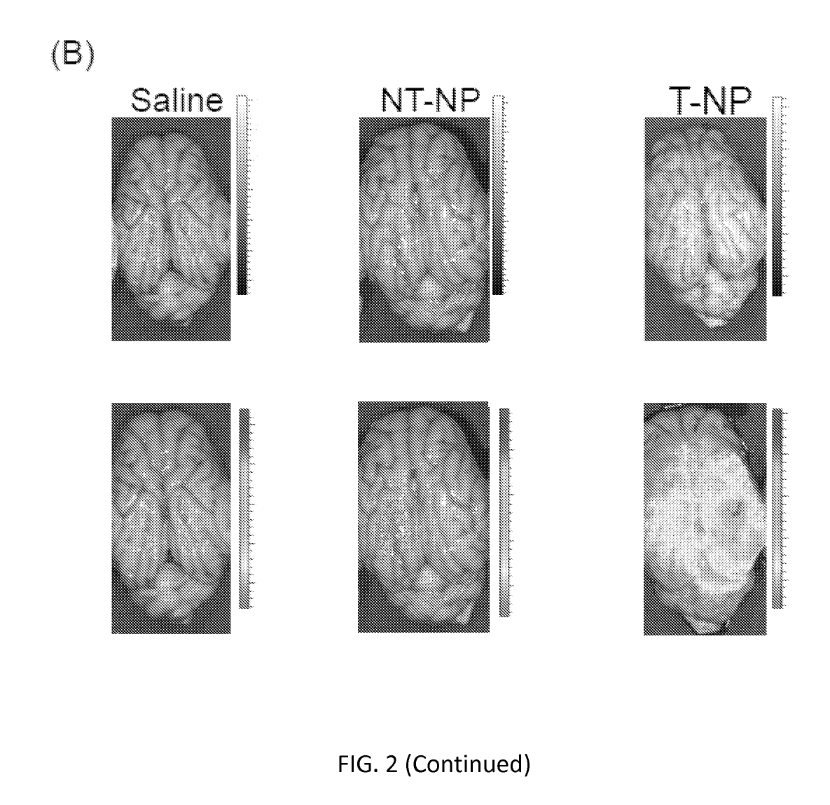

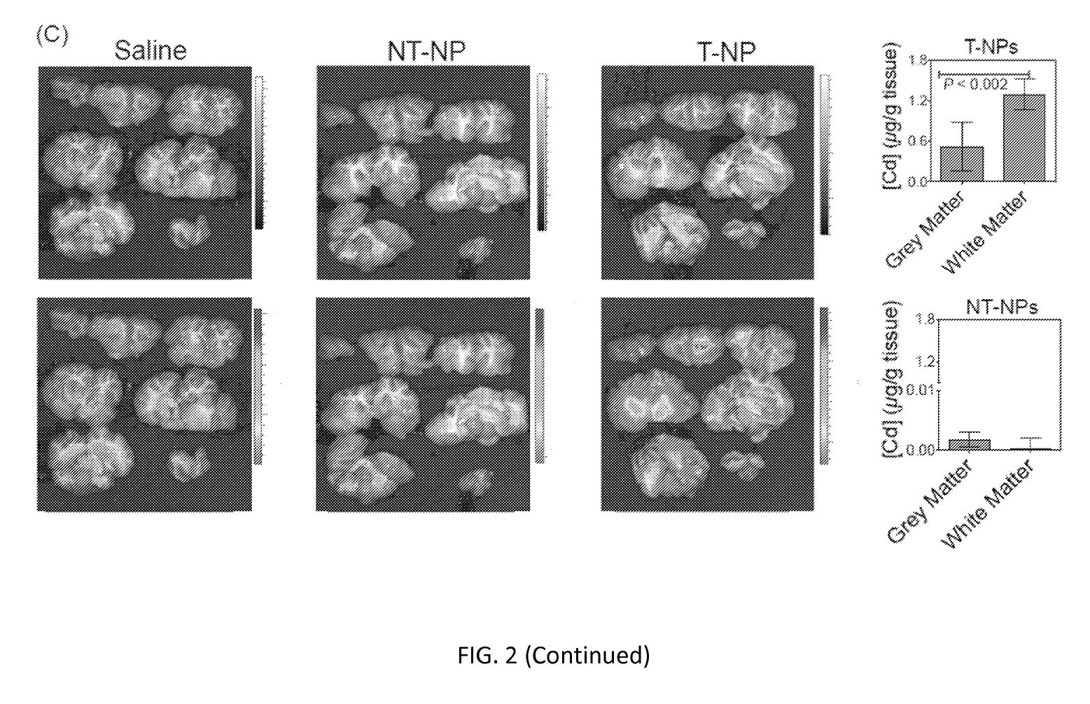

We recently determined brain accumulating properties of T-NPs using rat model. Since rodent brains are much smaller and anatomically different than human brains, it is difficult to assess whether the brain penetrating properties of T-NPs obtained in the rat is relevant to treatment of TBI in human. We therefore extended our analysis to piglets, which have larger brains. Targeted (T) and nontargeted (NT) NPs loaded with quantum dots (QD), T-QD-NPs (2.5 mg/kg with respect to NP and 0.46 mg/kg with respect to Cd), were administered via intravenous (i.v.) injection to American Landrace piglets. NT-QD-NPs (2.5 mg/kg with respect to NP and 0.62 mg/kg with respect to Cd) and saline were used as controls. Each group had three animals. NP size and zeta potential of the NPs used in this study are represented in Table 3. Blood samples were collected at predetermined time points after i.v. NP administration and amount of Cd present in the plasma were determined by inductively coupled plasma-mass spectroscopy (ICP-MS) (FIG. 2A, Table 1). The plasma Cd profiles of from T and NTQD-NPs were used to evaluate PK parameters (Table 1). Peak plasma concentration (Cₘₐₓ) was calculated directly from the time-concentration curves for QD. The fitted parameters did not differ significantly between the pigs treated with T-QD-NPs versus NT-QD-NPs (Table 1). The elimination half-life (t_{1/2}) was 2.3 h for T-QD-NPs that is in close agreement with the half-life previously observed in a rat model. A similar t_{1/2} value of 2.5 h was observed for NT-QD-NPs demonstrating that the positively charged TPP moieties do not have any effect on the clearance of the T-NPs. High area under the curve (AUC) values for blood concentrations were for T-QD-NPs and NT-QD-NPs. A smaller volume of distribution (V_{d}) was observed in the pigs receiving both TQD-NP and NT-QD-NP (Table 1). Animals were sacrificed after 24 h and bioD was studied by quantifying Cd in the different organ samples (FIG. 2A). The brain samples were analyzed by performing fluorescence microscopy using in vivo imaging system (IVIS) (FIG. 2B). Significant distribution of T-NPs was observed in pig brain, no such distribution in the brain was noted with the NT-NPs (FIG. 2B). NT-QD-NPs were mostly distributed in the liver (FIG. 2A).

TABLE 1
PK Profiles of T and NT-NPs in Pig Model

| | T-NPs | NT-NPs |
|---|---|---|
| Cd Does (mg/kg) | 0.46 | 0.62 |
| AUC_{[0-24 h]} (ng · h/ml) | 12,250 ± 715 | 12,617 ± 654 |
| Cₘₐₓ (ng/ml) | 3,666 ± 158 | 3,586 ± 410 |
| V_{d} (ml/kg) | 126.2 ± 5.3 | 174.7 ± 20.7 |
| C_{L} (ml/h) | 188.9 ± 11.1 | 246.0 ± 13.0 |
| T_{1/2} (h) | 2.32 ± 0.08 | 2.45 ± 0.17 |

Least-squares fit to model: C = A*exp(−k₁*t)

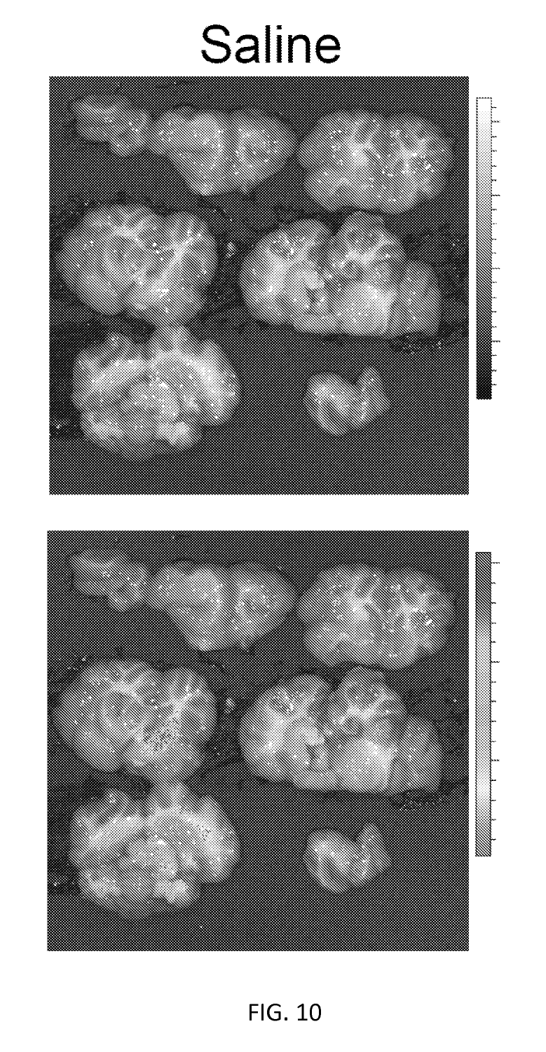

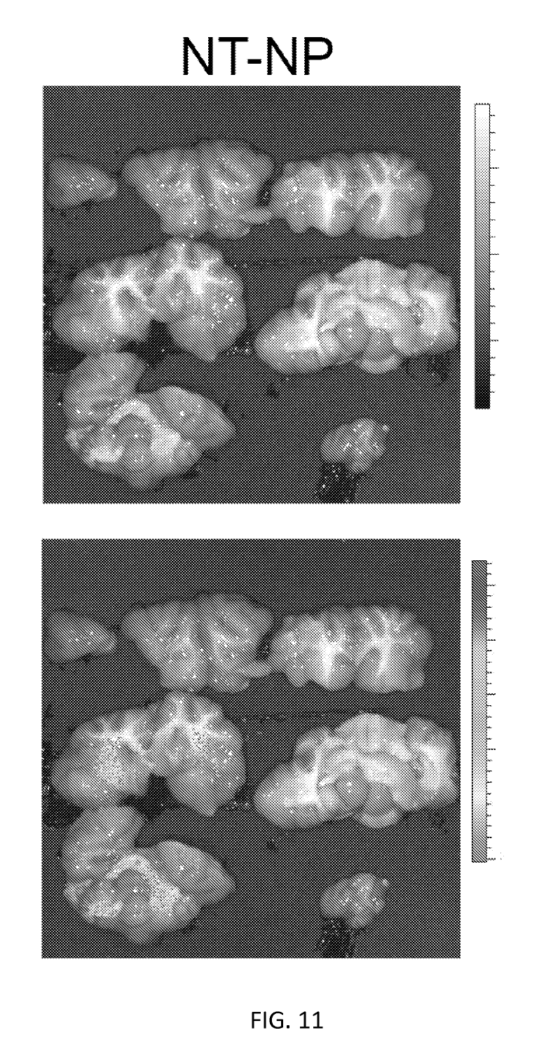

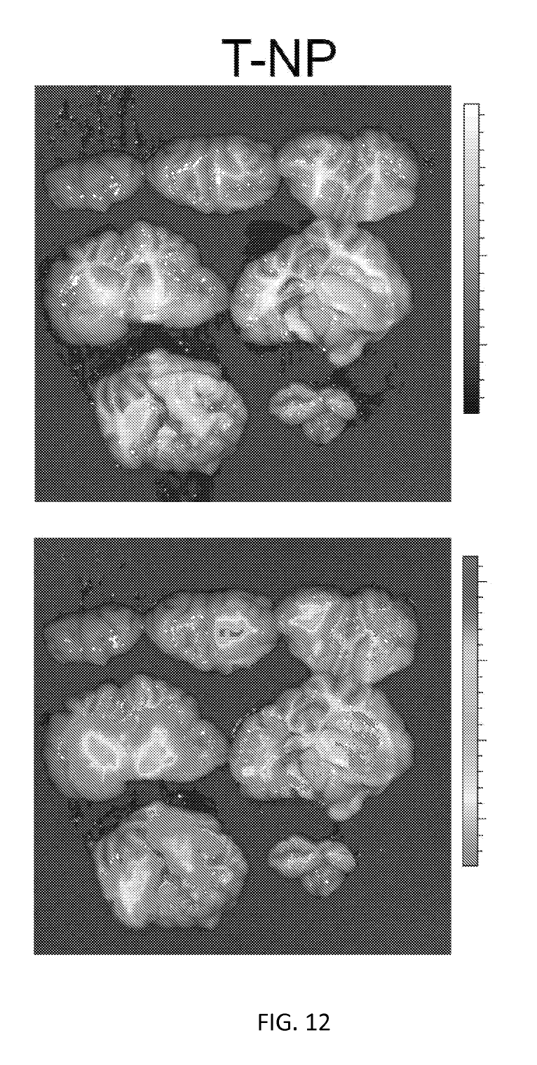

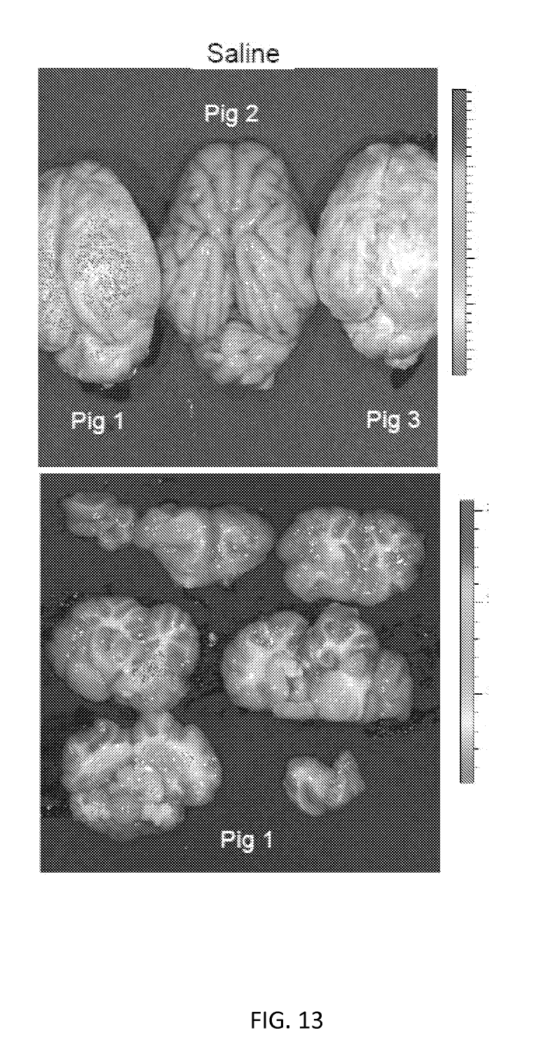

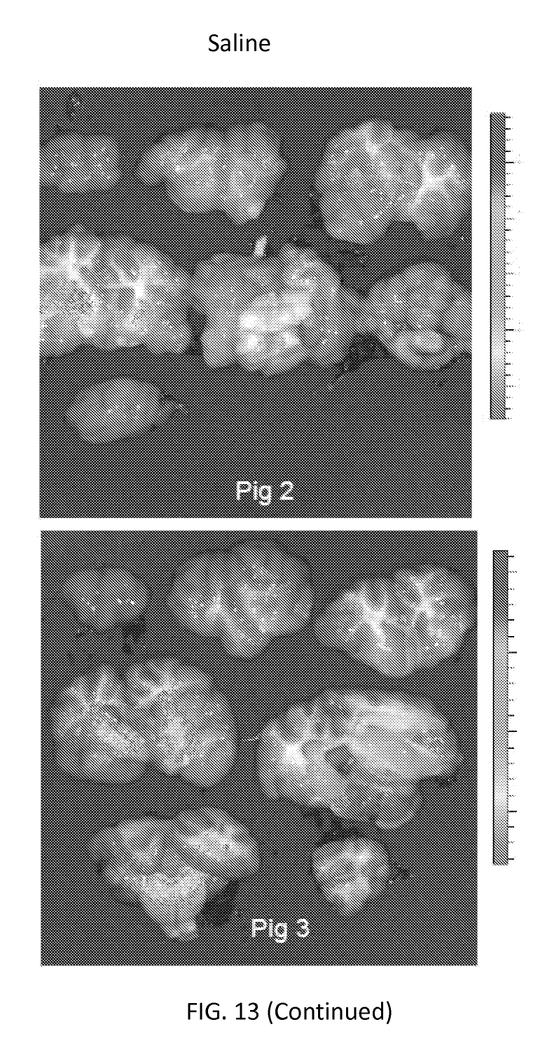

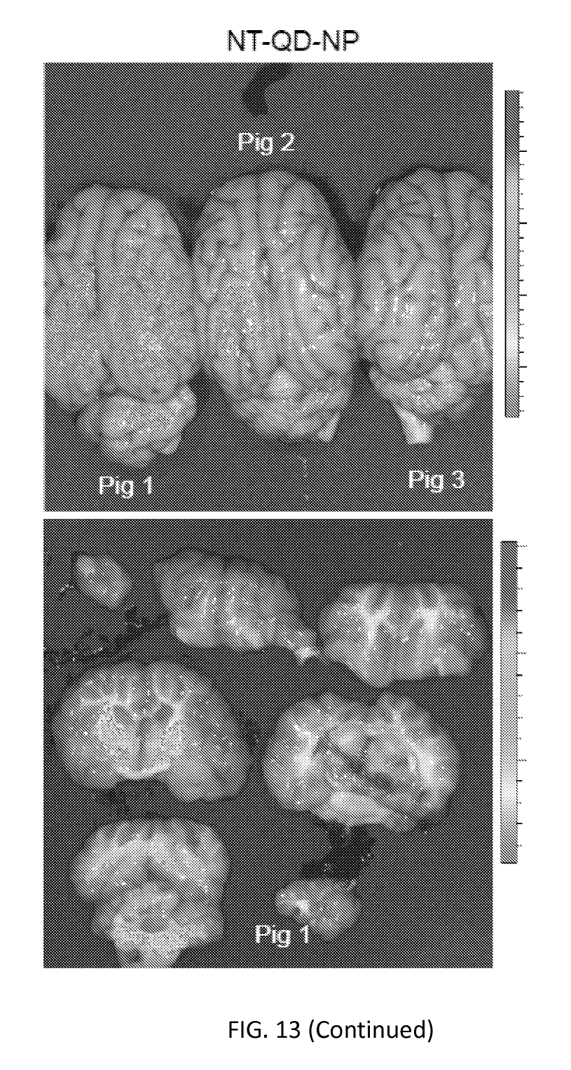

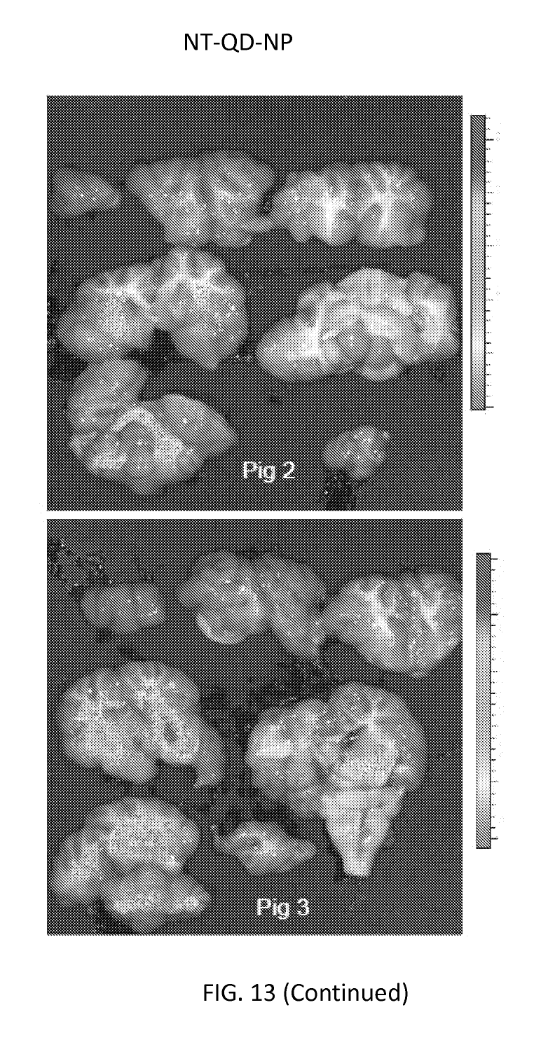

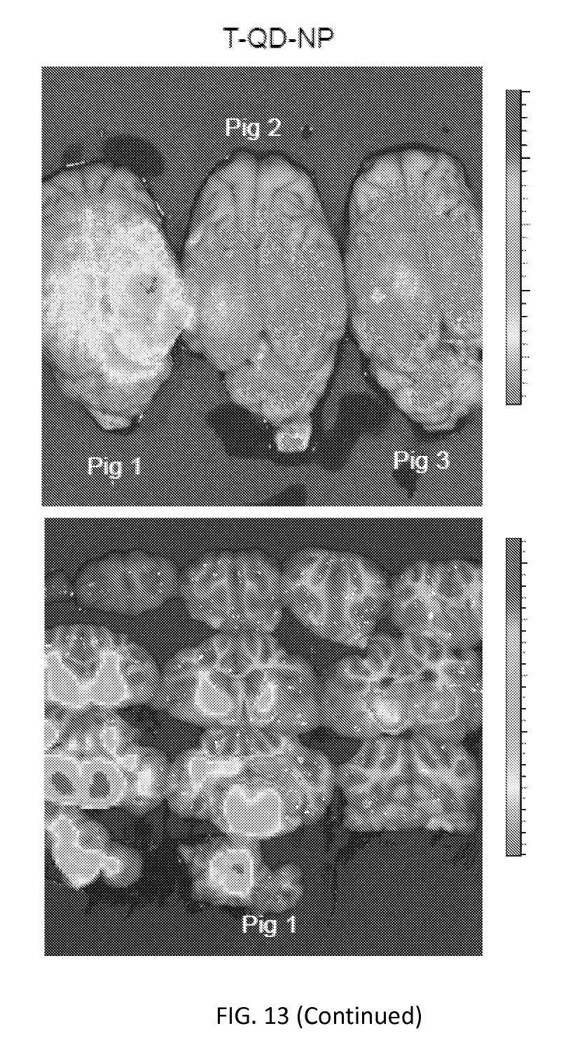

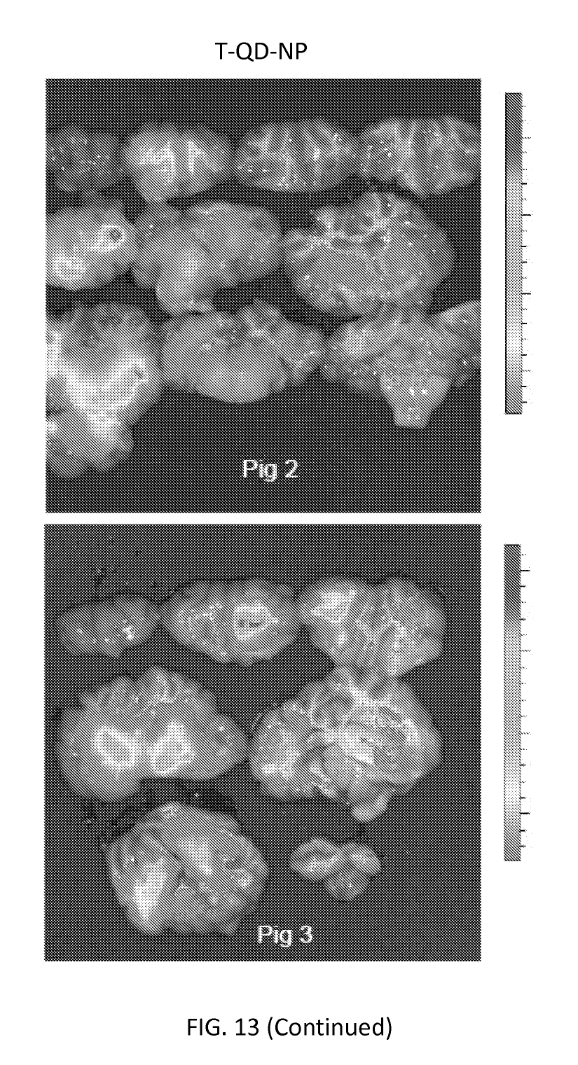

The brain is not a homogenous organ and the phospholipid pattern varies in its different regions resulting different lipophilicity profiles. Total lipid content in the white matter is twice as high as in the grey matter. The white matter with a higher total lipid content have higher levels of cerebrosides and sulfatides and lower percentages of phosphatidylcholine and phosphatidylinositol. IVIS analyses of sectioned brain slices indicated greater distribution of the T-NPs in the lipophilic white matter to a greater extent (FIG. 2C). This pattern was consistent across all animals studied (FIGS. 10-13). The greater distribution of T-NPs in the white matter was further confirmed quantatively by ICP-MS (FIG. 2C). Inflammation and injury are often diffuse in the white matter, therefore the current T-NPs which selectively accumulate in the white matter of the brain can be extremely beneficial in delivering neuroprotectants after TBI. Taken together, these data demonstrated that our brain-penetrating T-NPs have PK profile, distribution pattern, and in particular accumulation in brain white matter which are clinically relevant and suggest therapeutic potentials.

### Accumulation of T-NPS in the Mitochondria of Brain Tissue.

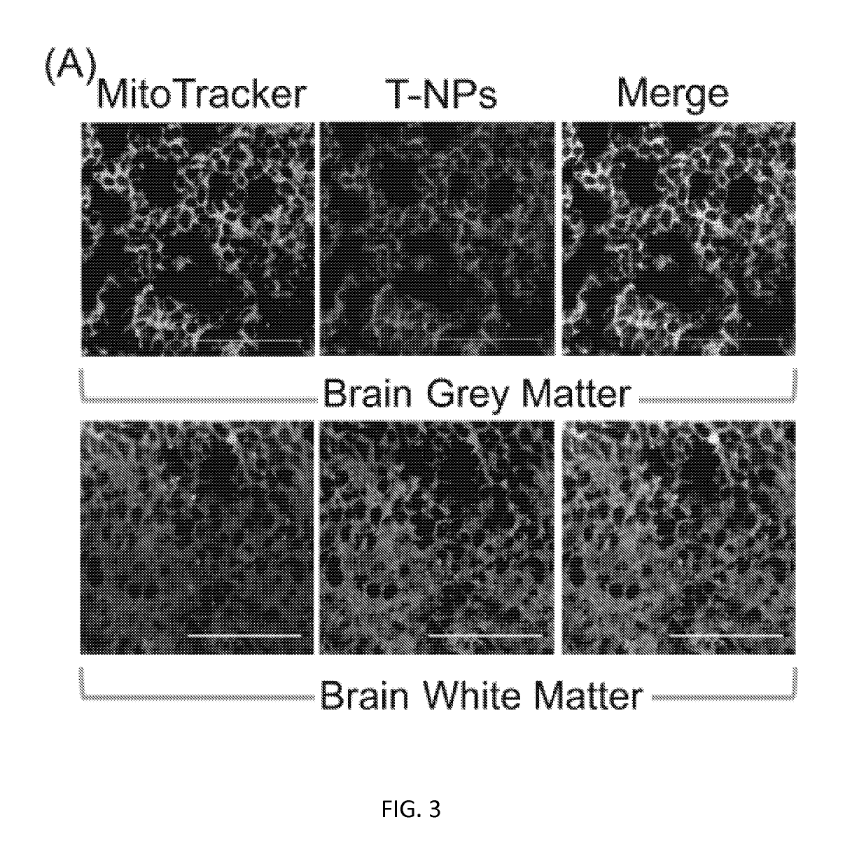

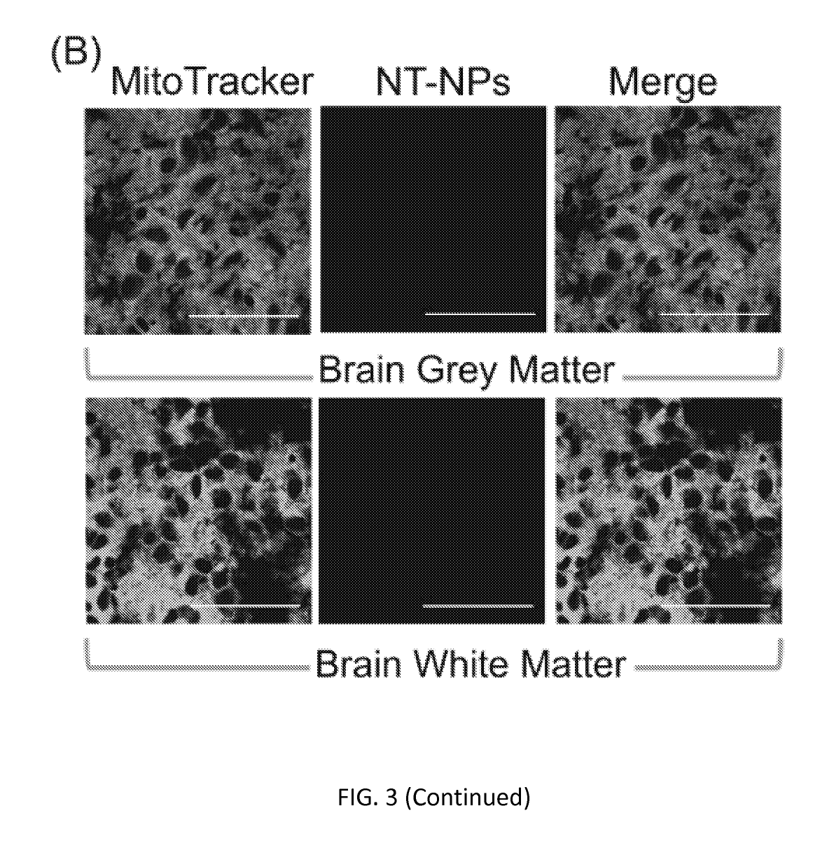

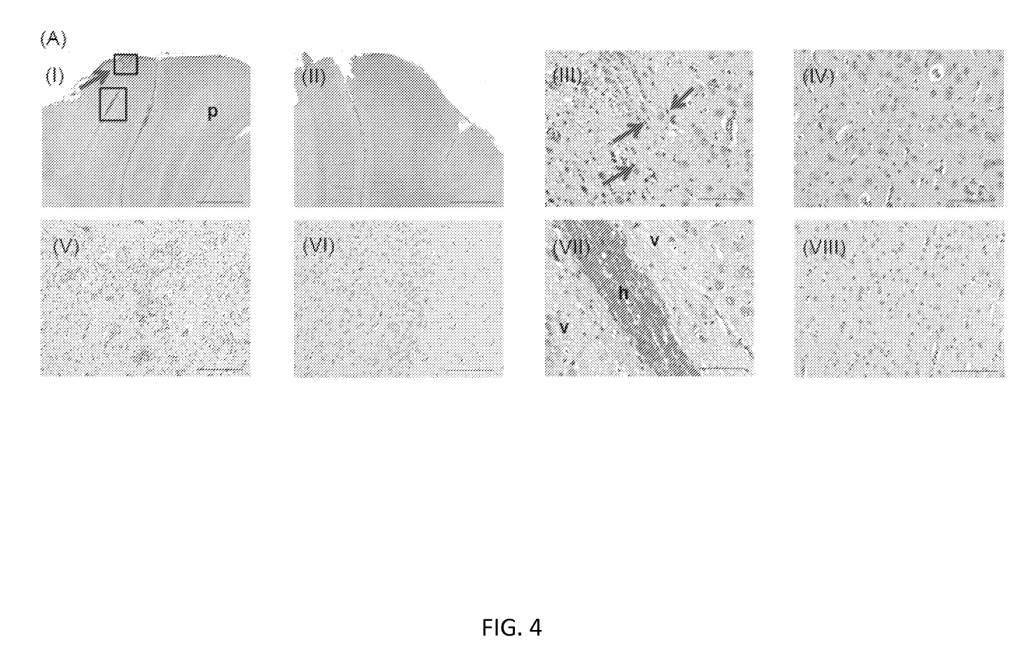

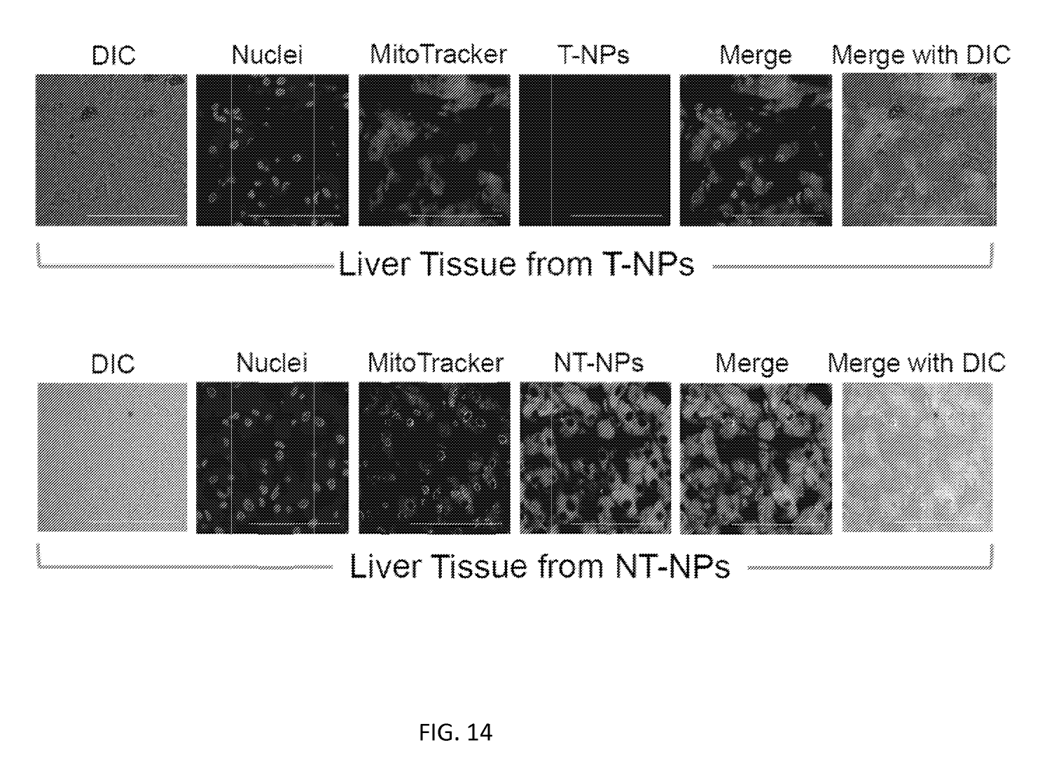

Because the T-QD-NP formulations showed significant accumulation in the brains and higher accumulation was observed in the lipophilic white matter, we subjected brain tissue samples from white and grey matter to additional confocal imaging (FIG. 3). Both gray and white matter samples from pig brains treated with T and NT-NPs were isolated, samples were fixed, mitochondria were stained with MitoTracker green, and the samples were sectioned for imaging. Confocal imaging of these samples illustrated significant association of T-QD-NPs in the mitochondria of brain cells present in the white and grey matters. Mitochondrial association of T-QD-NP was higher in the white matter compared to that in the grey matter. The NT-QD-NPs were not detected in the brain tissue samples (FIG. 3B). NT-QDNPs were randomly distributed in the liver cell cytoplasm (FIG. 14), however only limited T-QD-NPs were found in the liver cells (FIG. 14).

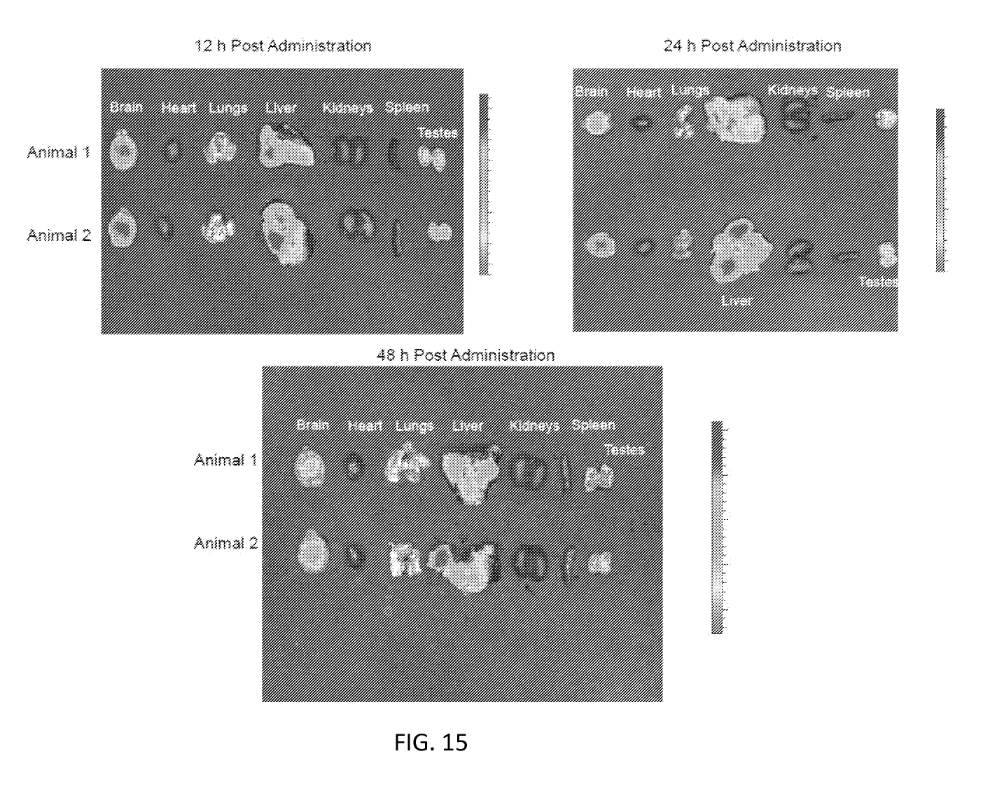

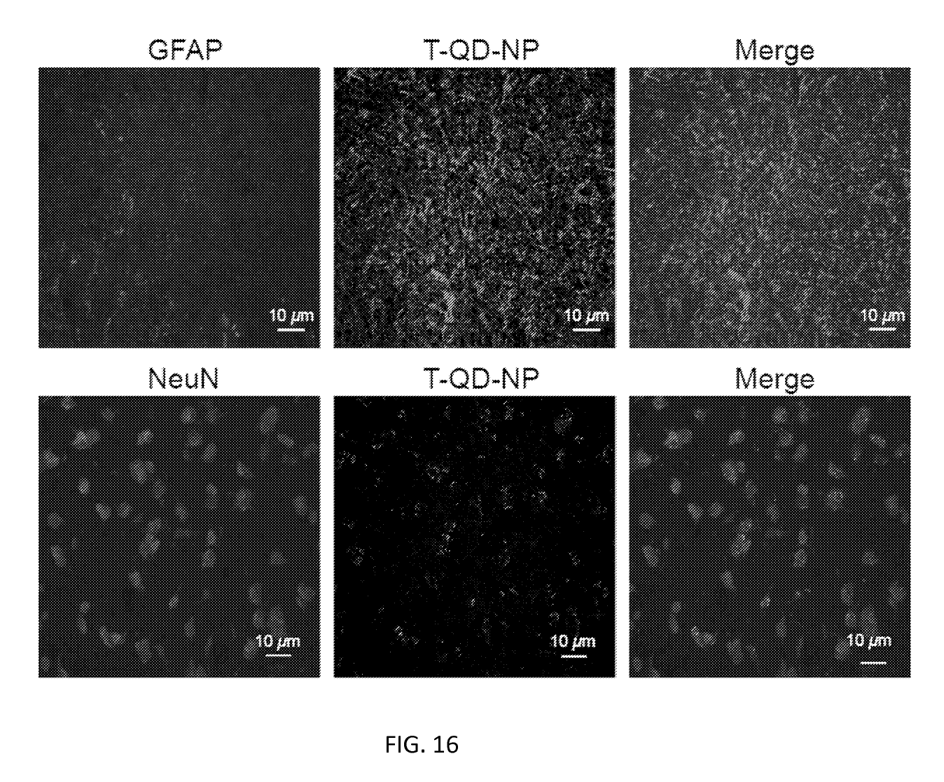

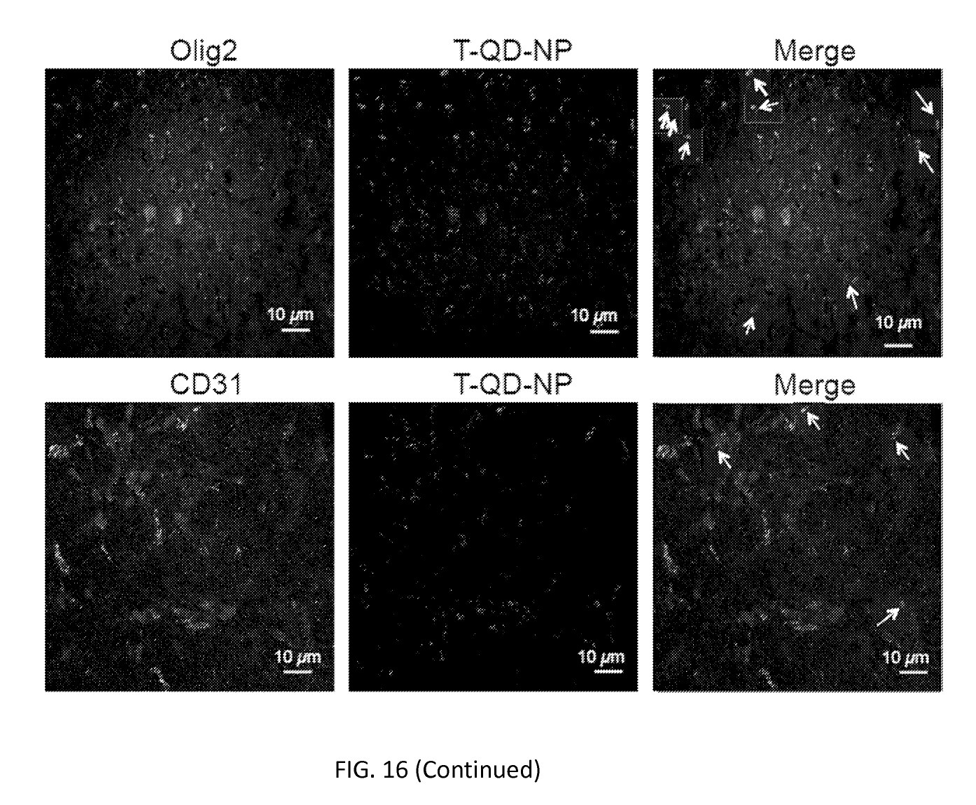

These results demonstrate that TNPs have a high affinity for mitochondria, which will lead to improved targeting of antioxidant neuroprotectants. To understand the distribution of T-NPs further, we performed time-dependent accumulation of these NPs in healthy mice by i.v. administration. Three different time points of 12, 24, and 48 h were studies and brain accumulation of T-QD-NPs were compared by IVIS imaging at these three time points (FIG. 15). A comparison of QD emission intensities in the brains of the treated samples indicated that the NPs accumulate as early as 12 h and stay in the brain even after 48 h. Microscopic analyses were performed on the brain samples from animals treated with T-QD-NPs for 12 h. Immunostaining of brain tissue sections were performed using antibody treatments against different types of brain cell markers: NeuN for neuronal nuclei, CD-31 for normal endothelial marker, olig2 for oligodendrocytes, and GFAP for astrocytes (FIG. 16). These studies indicated that the TQD-NPs have preferential association with the oligodendrocytes and endothelial cells over the neurons and astrocytes (FIG. 16). These studies further confirmed that the T-QD-NPs are taken up by the brain cells.

### Development of TBI in Pig.

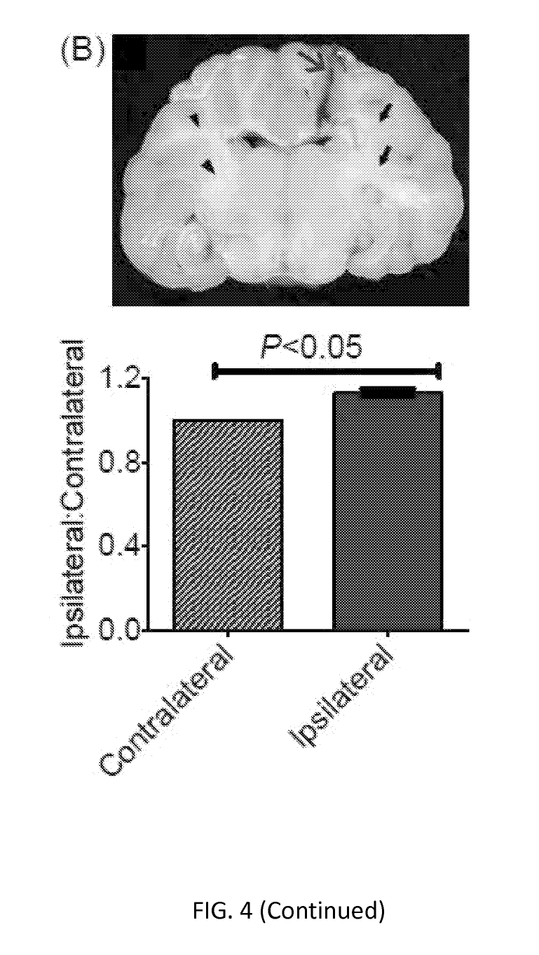

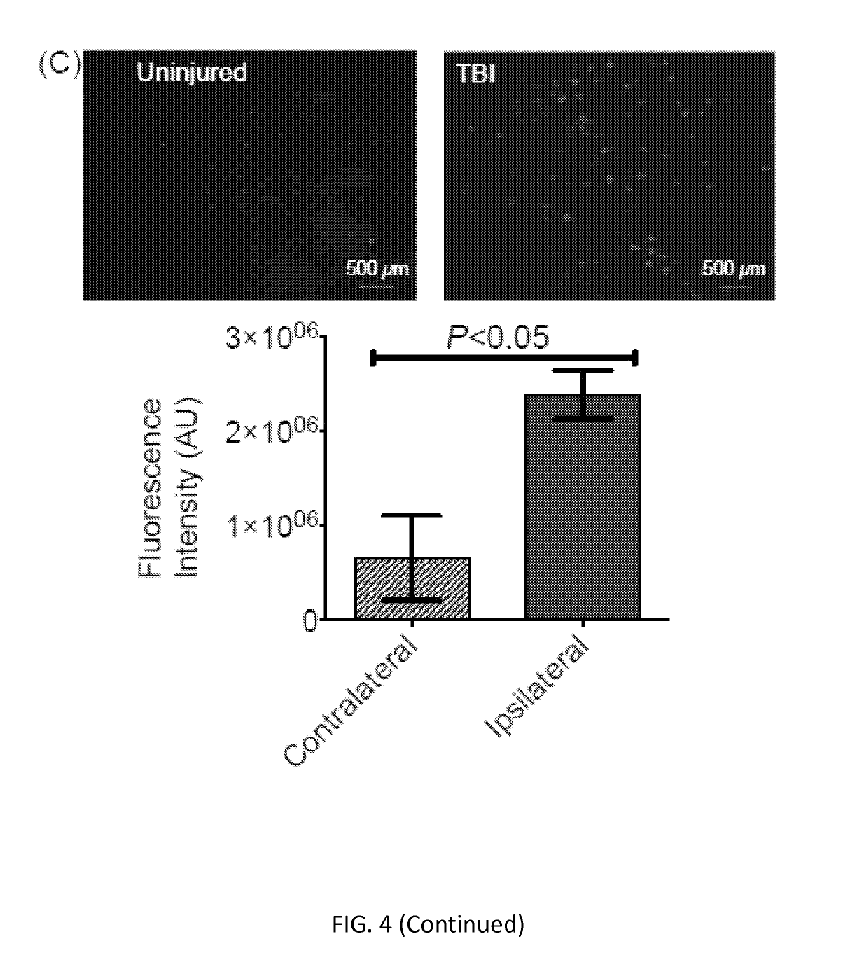

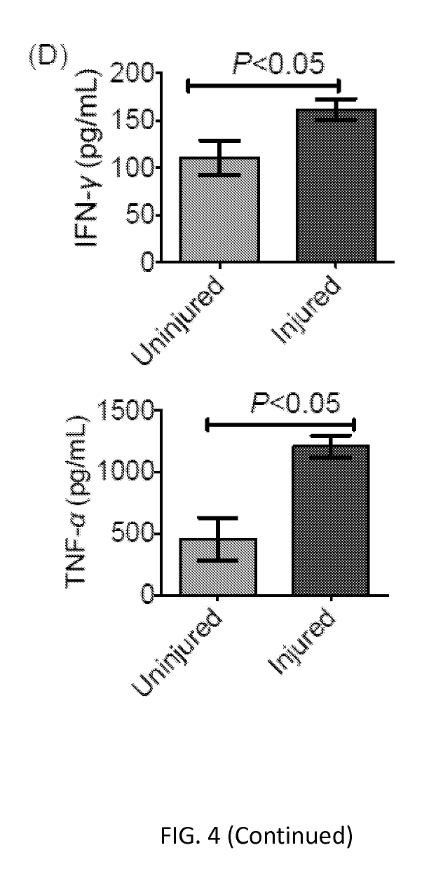

One of the difficulties in developing effective treatments for TBI is the poor translatability of therapies from rodents to human patients. Compared to the rodent, the piglet brain has greater anatomical and physiological similarities to humans. This suggests that the piglet TBI model may be more advantageous than the widely used rodent model for neural injury as outcomes are likely to be more predictive of what would occur in an immature human brain. The piglet has a comparable neurodevelopmental sequence to humans. The postnatal maturational sequence shows that the porcine species has a similar shape, gyral pattern, and grey to white matter ratio as humans. Whereas the rodent cerebral cortex is lissencephalic, the surface of pig brain more closely resembles the human gyrencephalic neocortex. Both the human brain is composed of more than 60% white matter, while the rodent brain contains less than 10% white matter. The brain of larger animals such as the pig contains more white matter than that of the rodent, and this is important because of the differences in blood flow, metabolism, and injury mechanisms in white versus grey matter. We therefore, used TBI in pig model in our studies. Detailed descriptions of generation of TBI in pig are discussed in the method section. The TBI resulted in significant changes in the brain at both the cellular and tissue levels (FIG. 4). Hematoxylin and eosin staining revealed presence of considerable hemorrhage in the brain parenchyma of the ipsilateral hemisphere 24 h after the onset of injury (FIG. 4A-I). This was accompanied by edema accumulation in the white matter at the junction of the cortex; this is likely vasogenic edema resulting from increased vascular permeability due to breakdown of the BBB commonly encountered in TBI. The affected cortex contained dying neurons represented by blue arrows that were specific to the ipsilateral hemisphere as the contralateral cortex displayed normal morphology (FIG. 4A-III and FIG. 4A-IV). Additionally, the affected cortex showed neutrophil invasion (FIG. 4A-V). These inflammatory cells are recruited across the BBB from the periphery in response to damage-associated molecular patterns (DAMPs) and pro-inflammatory cytokines. The presence of neutrophils in the brain parenchyma further evidenced inflammation and disruption of the BBB. In the white matter region of the ipsilateral hemisphere, considerable hemorrhage was present and edema accumulation was confirmed by high levels of vacuolation (FIG. 4A-VII). This observation coincided with previous findings that vasogenic edema predominantly accumulates in the interstitial spaces of white matter because the dense meshwork of gray matter neuropil is resistant to interstitial edema. Edema accumulation was correlated to an overall increase in area of ipsilateral hemisphere relative to the contralateral hemisphere (FIG. 4B). Black arrows highlighted arrows of distended white matter on the ipsilateral hemisphere compared to the black arrowheads on the contralateral hemisphere. The area of the ipsilateral hemisphere was on average 13% greater than the respective contralateral hemisphere (FIG. 4B).

In addition to pathophysiological alterations at the tissue levels, pathology was apparent at the cellular levels. Tissue at the lesion site of the ipsilateral hemisphere as well as the contralateral hemispshere underwent analysis to detect ROS presence, an indicator of excitotoxicity. There was a significant increase in ROS at the lesion site relative to the corresponding region of the contralateral hemisphere (FIG. 4C). Furthermore, the amount of pro-inflammatory cytokines, which are important mediators of inflammation in neural injury, were measured in both injured and uninjured animals. There was a significant up-regulation of both interferon-gamma (IFN-γ) and tumor necrosis factor-alfa (TNF-α) protein levels in injured animals compared to uninjured pigs (FIG. 4D). These results indicated a substantial neural injury that is associated with neural cell death, excitotoxicity, and inflammation.

### Distribution of T-NPs in Piglet Model of TBI.

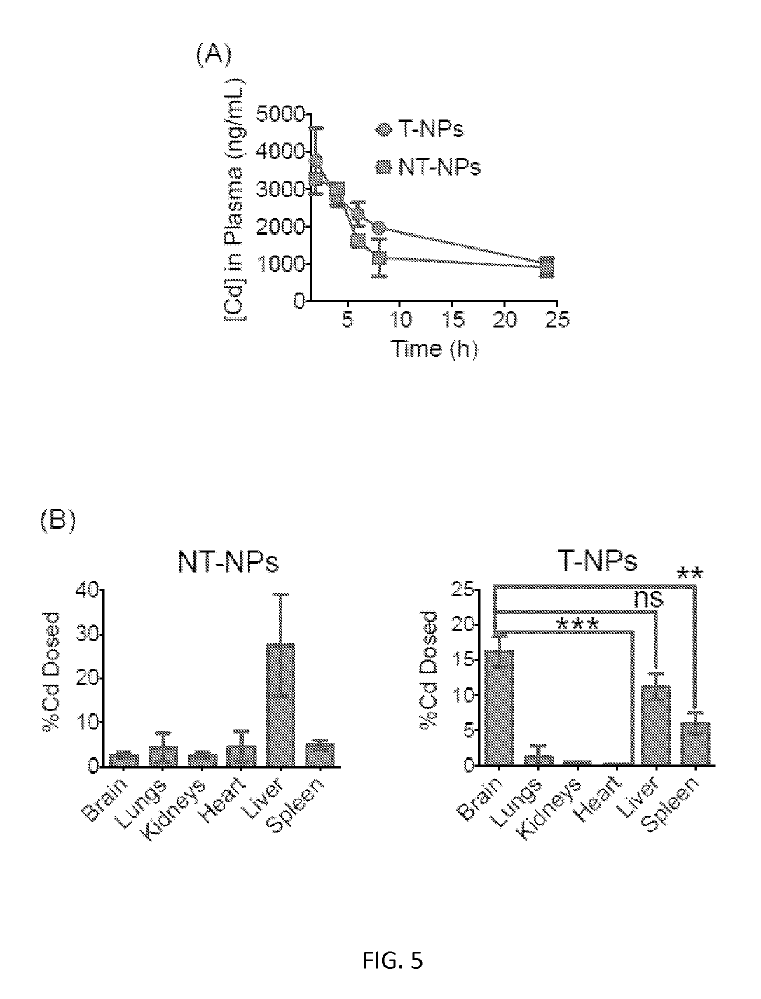

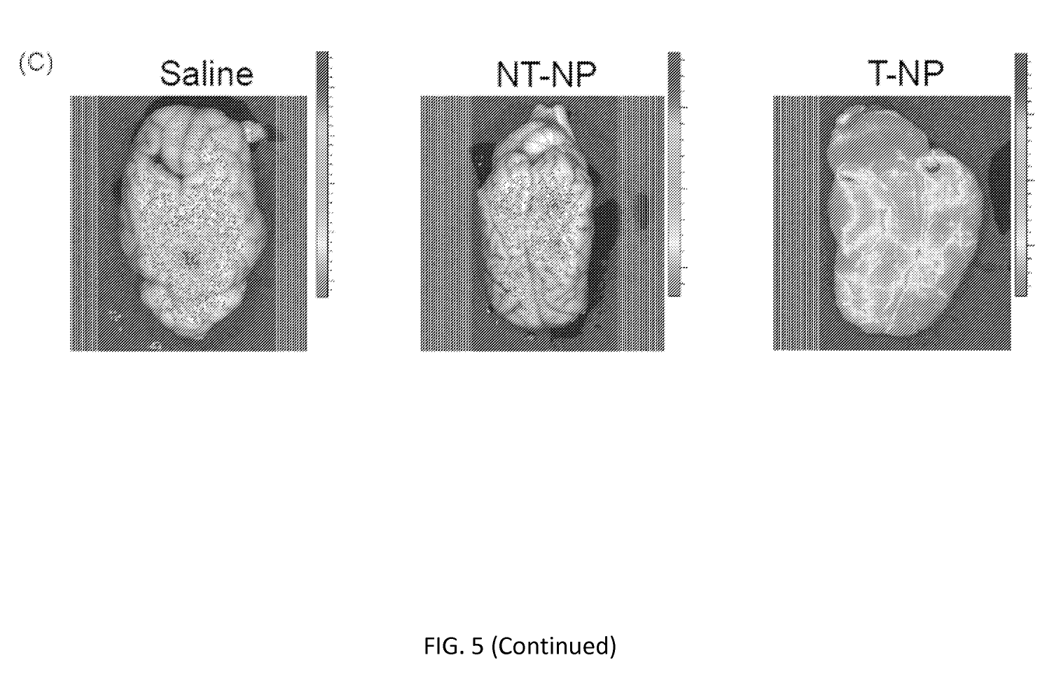

We next assessed distribution properties of T-QD-NPs and NT-QD-NPs in TBI pig model. A penetrating TBI was generated 5 h prior to intravenous NP injection. T-QD-NPs and NT-QDNPs were administered by ear vein injection. In contrast to studies in normal pig, the PK parameters for T and NT-NPs differ significantly when evaluated in injured animals (FIG. 5 and Table 4). There was significant difference between the plasma t₁/2 values for T-QD-NPs compared to NT-QD-NP treatments. Notable in these results are increase in the AUC for the TNPs over the NT-NP formulation. This coupled with the observation that the T-NPs has a greater mean residence time in the plasma (t_{1/2}: 9.6 h compared to t_{1/2} of NT-NPs: 5.7 h), is a confirmation of unique features of TPP containing T-NPs that allow it to circulate for longer times. Organ distribution indicated a major fraction of T-QD-NPs in the brain and most of NTQD-NPs was found in the liver (FIG. 5B). The preferential distribution of the T-NPs in the brain was further confirmed by performing IVIS on the injured brain samples (FIG. 5C). The T-NPs were present in the brain at a much higher levels than NT-NPs. The PK and distribution profiles of T-NPs demonstrated that mitochondria targeted NPs can be extremely beneficial in delivering neuroprotectants after TBI. We believe that the similar distribution pattern of T-NPs in both non-injured and injured animals due to the fact that a significant portion of the BBB remains intact since the injury is mild and highly targeted (15 mm×10 mm×10 mm). This type of injury is significantly different than an injury that would occur from global ischemia, which would lead to breakdown of the BBB at numerous locations.

### Reduction of Inflammation and Oxidative Stress in Neural Stem Cells (NSCs).

Transplantation of NSCs can provide a promising therapy after TBI. However, the efficacy of such NSC transplantation is limited because of massive grafted-cell death and insufficient tissue repair. Stem cells in the nervous are NSCs that can renew and differentiate to differentiated progenitor cells for generation of lineages of neurons and glia. We therefore, used NSCs to study the therapeutic potential of BBB penetrating T-NPs containing prednisone and CoQ₁₀.

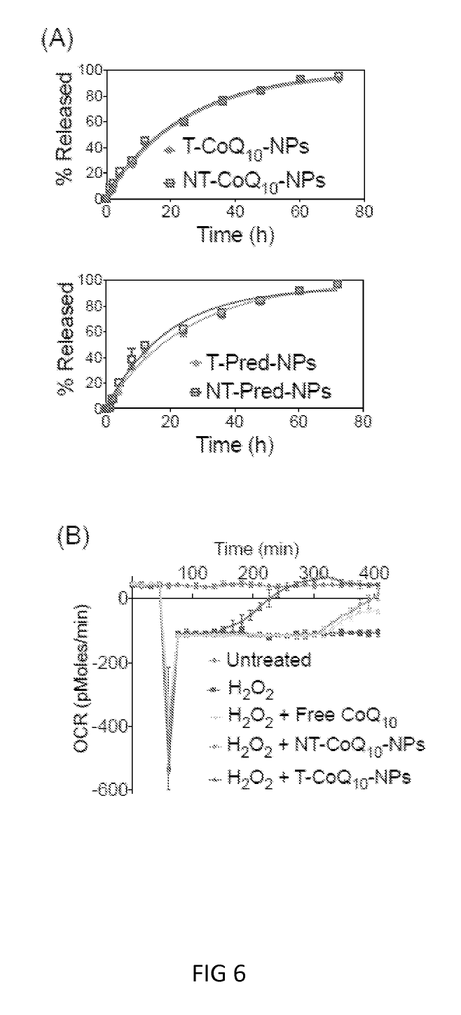

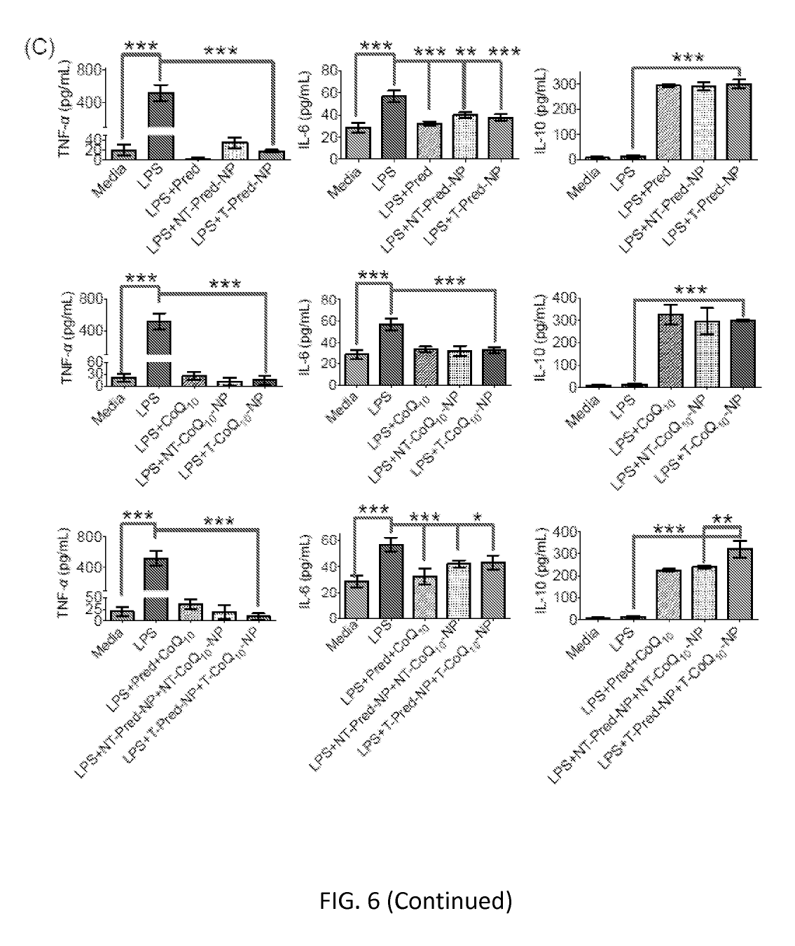

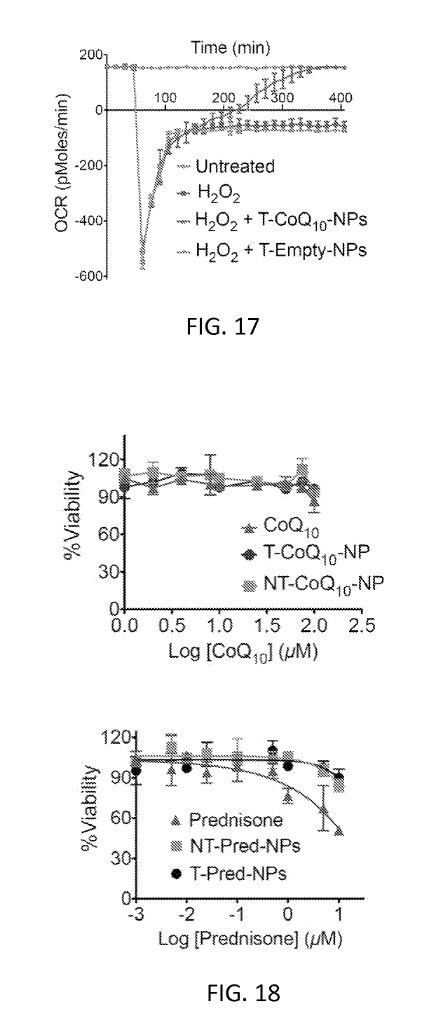

Oxidative stress induced by the production of ROS including free radicals and peroxides is one of the major mechanisms, which leads to neuronal destruction and is closely related to apoptosis and necrosis in these cells during TBI. Mitochondria are well known to be a major source of ROS production. CoQ₁₀ is endogenously synthesized in mammalian mitochondria, acts as both antioxidant and pro-oxidant, and is involved in shuttling electrons from complexes I or II and a number of other electron donors, including electron transfer factor, which moves electrons from fatty acid beta oxidation. CoQ₁₀ is found in all cell and organelle membranes, where it can participate in redox shuttling. However, defects in CoQ₁₀ biosynthesis can be found during TBI. Therefore, exogenous CoQ₁₀ administration to brain during TBI can represent an attractive strategy to reduce local oxidative stress. However, CoQ₁₀ is insoluble in water, powder formulations have very poor intestinal absorption, and no BBB penetrating properties. Improved bioavailability, controlled release, brain accumulation, and efficient mitochondrial distribution of CoQ₁₀ using T-CoQ₁₀-NPs can be extremely beneficial for TBI treatment. Both T-CoQ₁₀-NP and NT-CoQ₁₀-NPs demonstrated controlled release of the antioxidant under physiological conditions (FIG. 6A). We induced oxidative stress in NSCs by bolus doses of hydrogen peroxide (H₂O₂) and studied the effect of mitochondria-targeted T-CoQ₁₀-NPs on mitochondrial respiration using a oxygen consumption rates (OCRs). Addition of H₂O₂ to the NSCs decreased OCRS significantly (FIG. 6B). Subsequent addition of T-CoQ₁₀-NPs to H₂O₂ treated NSCs demonstrated reversal of the oxidative stress. In comparison NT-COQ₁₀-NPs and free CoQ₁₀ showed less efficiency in oxidative stress reduction and the OCR levels were less than those observed in healthy cells. A control experiment using T-Empty-NPs (0.5 mg/ml) did not show any significant oxidative stress reduction (FIG. 17). Taken together these results demonstrated a decreased OCR in H2O2-treated NSCs. Mitochondrial delivery of CoQ₁₀ in the form of T-CoQ₁₀-NPs demonstrated remarkable efficiency in reducing H₂O₂ mediated oxidative stress in NSCs.

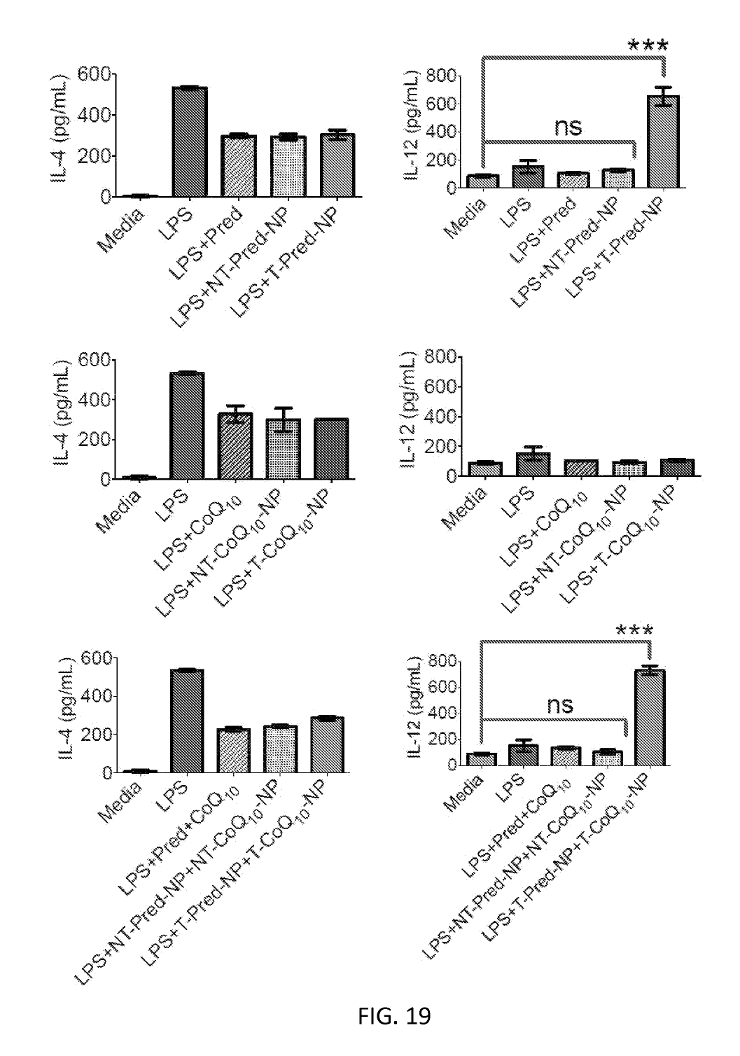

Immunosuppressant corticoid based rugs such as prednisone can exert biphasic effects on neuronal mitochondrial dynamics. A low level of prednisone and chronic high levels can attenuate various aspects of mitochondrial function. We therefore hypothesized that by delivering prednisone in a controlled release fashion using T-Pred-NPs as evident by the release kinetics showed in FIG. 6A to the mitochondria of NSCs, we can potentially observe effects related to mitochondrial oxidation, membrane potential, and calcium holding capacity. Inflammatory cytokines like TNF-α and IL-6 are closely linked to TBI and these cytokines have a negative effect on damaged tissue. Therefore limiting inflammatory cytokine activity is of major benefit in reducing TBI tissue damage. We used lipopolysaccharide (LPS) to stimulate TNF-α and IL-6 production in NSCs to understand the anti-inflammatory effects of prednisone-NPs and CoQ₁₀-NPs in an in vitro model (FIG. 6C). When LPS stimulated NSCs were treated with prednisone, T-Pred-NPs, NT-Pred-NPs, T-CoQ10-NPs, NT-CoQ10-NPs or any combination of the T and NT-NPs, TNF-α secretion induced by LPS was significantly reduced. IL-6 levels in cerebrospinal fluid can be significantly higher than plasma levels in patients who had suffered TBI. The T and NT NPs containing prednisone reduced the levels of IL-6 in the LPS stimulated NSCs. The addition of T or NT-CoQ10-NPs to prednisone NPs increased the efficiency of IL-6 reduction. The concentrations for prednisone or CoQ10 used in these studies did not show any cytotoxic effects cytotoxic effects in the treated NSCs ruling out the possibility of cell death (FIG. 18). We did not observe any difference between T, NT treated groups in reducing TNF-α and IL-6 under the in vitro settings. IL-10 is primarily an anti-inflammatory cytokine with potent inhibitory effects on several pro-inflammatory mediators. In our experiments, we found that IL-10 was elevated in NSCs when the cells were treated with CoQ10 or prednisone and their NPs. A combined administration of T-CoQ10-NP and T-Pred-NP was more effective in inducing IL-10 compared to the other formulations (FIG. 6C). IL-4 plays major roles as a negative regulator of pro-inflammatory cytokine production by both brain cells and T lymphocytes.38 All test articles showed a decreased IL-4 response when compared to LPS (FIG. 19). IL-12 has immune-inflammatory responses in the brain; however the consequences of local production of IL-12 on spontaneous immune responses are unknown. In our studies LPS treated NSCs that were stimulated with mitochondria targeted TPred-NPs or a combination of T-Pred-NPs and T-CoQ10-NPs showed significant increase in the IL-12 levels (FIG. 19). The exact mechanism why only mitochondria targeted therapy produces IL-12 from the NSCs warrants further investigation.

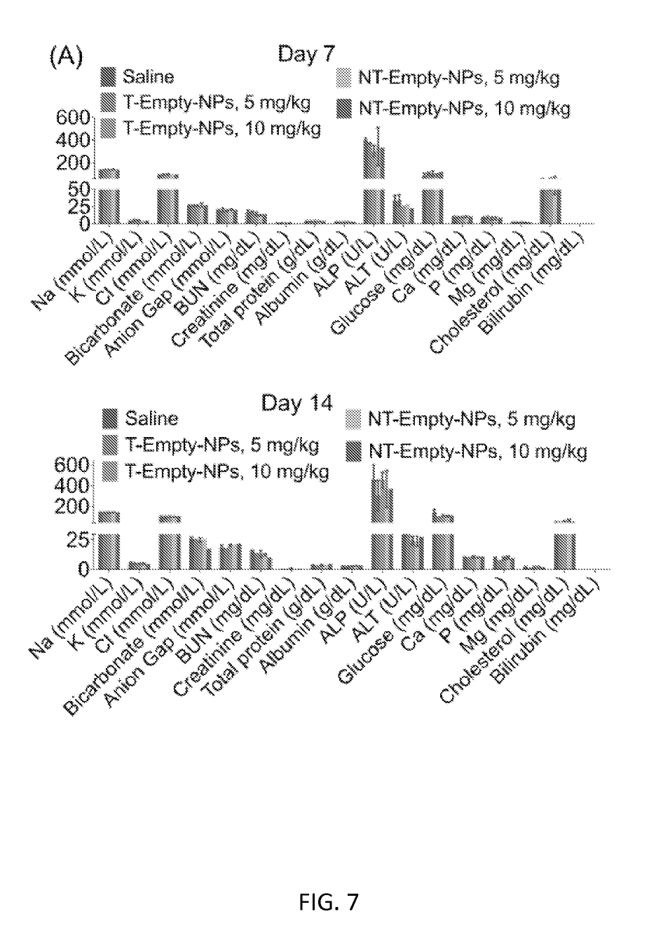

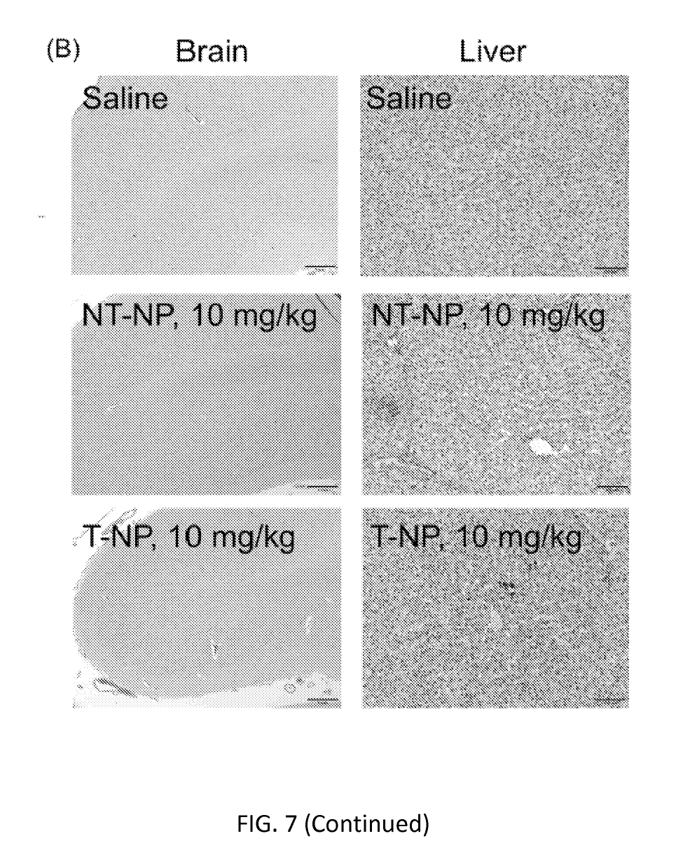

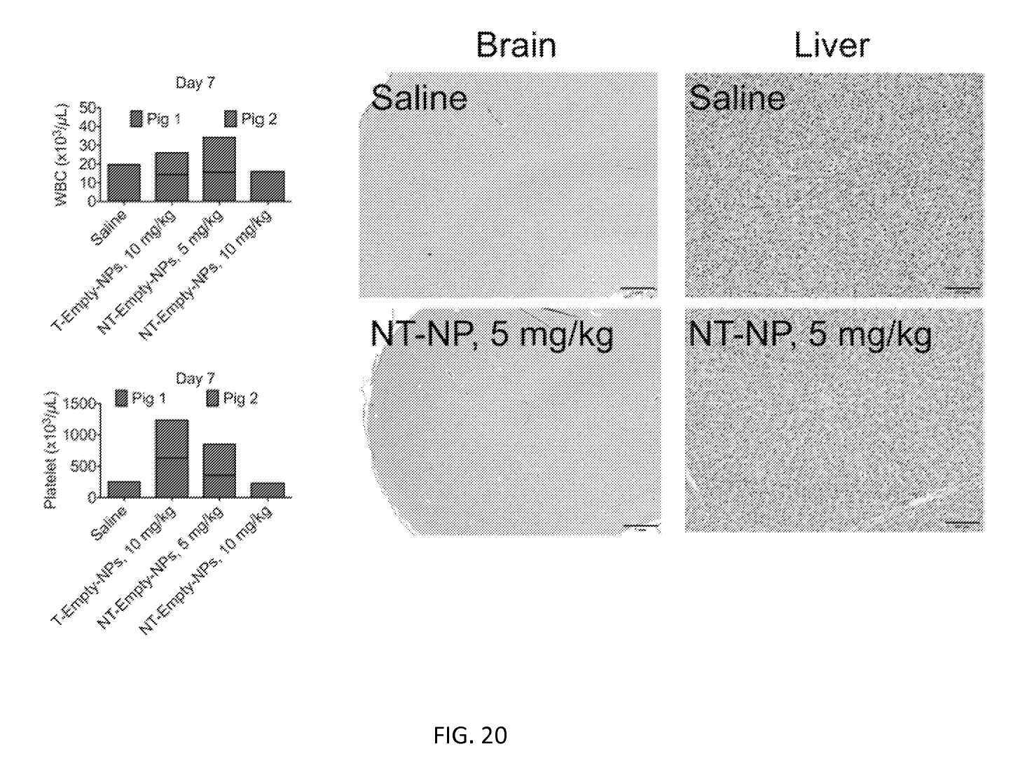

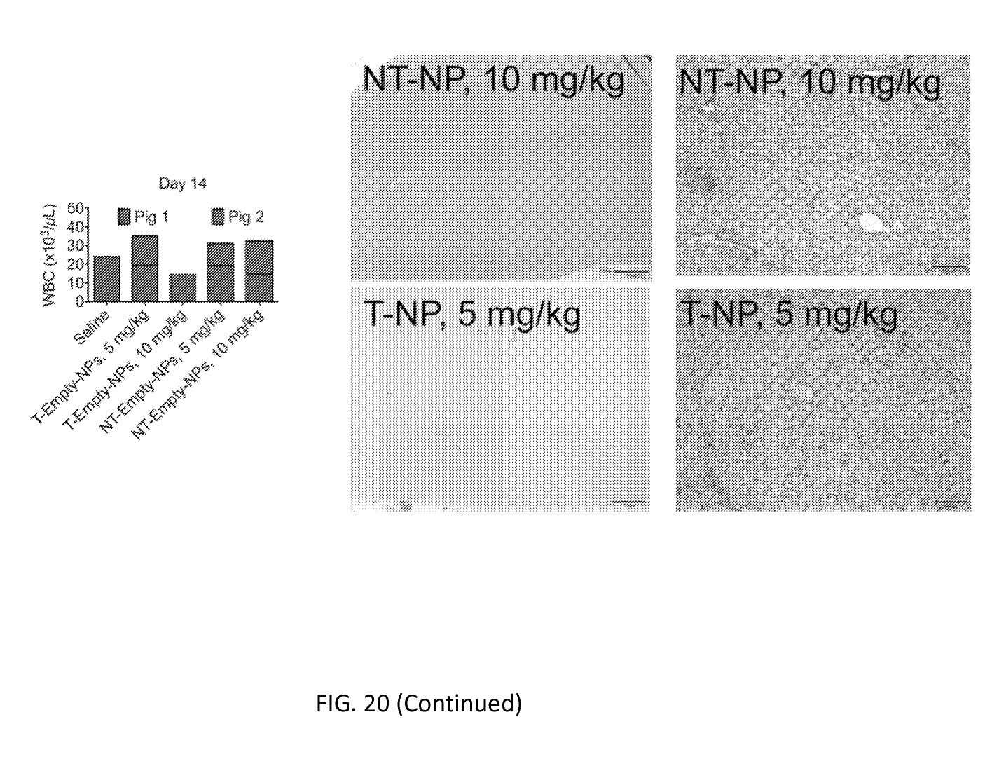

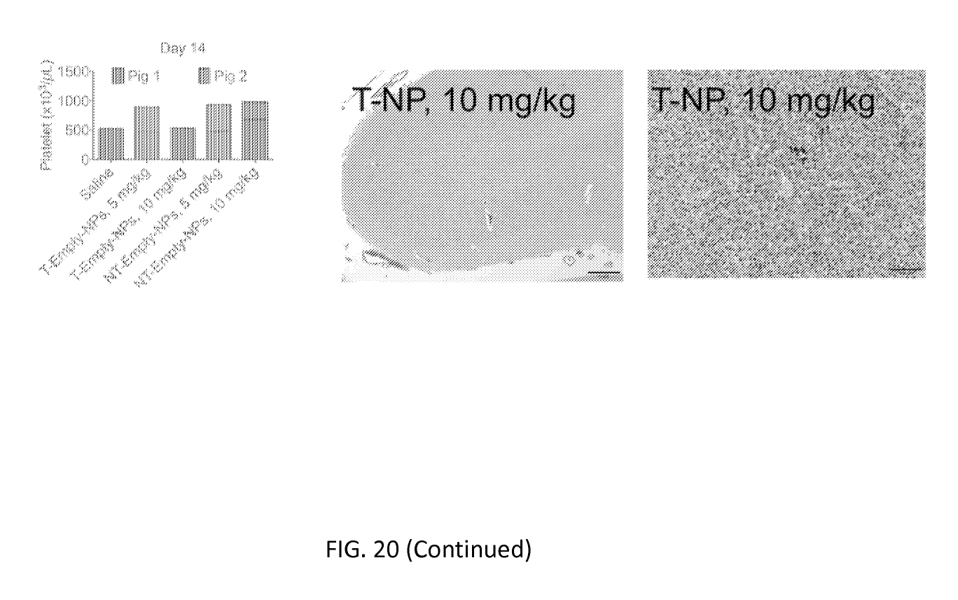

After demonstrating in vivo distribution and in vitro efficacy of this NP platform for brain injury, we next assessed safety of the T-NPs in pigs. A dose dependent 14-day toxicity and safety study was carried out in piglets in presence of T-Empty-NPs and the results were compared with NT-Empty-NP and saline treated animals. Two healthy piglets in each group was treated with TEmpty-NPs (5 mg/kg or 10 mg/kg with respect to total NP), NT-Empty-NPs (5 mg/kg or 10 mg/kg with respect to total NP), or saline by a single dose administration via ear vein. Serum clinical chemistry data from day 7 and day 14 shown in FIG. 7A indicated that most of the values were within clinically acceptable limits during the course of the study. No neurological or behavioral changes were observed during the study period. The only abnormality was one piglet receiving 5 mg/kg T-Empty-NPs and both piglets receiving 10 mg/kg T-Empty-NPs stopped breathing for 1-2 min during injection. However, after completion of injection and turning off isoflurane anesthetic, the piglets began breathing and recovered normally. “Starter 1” diet produced by the University of Georgia feed mill was fed ad libitum, and piglets gained weight at a normal rate. Assessment of standard hematologic parameters such as platelet white blood cell (WBC) count indicated no statistically significant difference between the T-NP treated animals with those treated with NT-NPs or saline (FIG. 20). This indicated that dose dependent administration of the T-Empty-NPs did not induce significant changes. Since T-Empty-NPs mostly distribute to the brain and liver and NT-Empty-NPs accumulate in the liver, we performed histological analyses of these organs. A section of well-fixed liver and unilateral sections of brain were sampled as described by Bolon et al., Toxicol. Pathol., 2013, 41, 1028-1048 for sampling and processing the nervous system during nonclinical general toxicity studies. In the liver, all pigs had scattered small foci of hematopoiesis, either erythropoiesis or erythro- and granulopoiesis. Given the age of the animals, hematopoiesis in liver, referred to as extramedullary hematopoiesis, is likely normal and residual from that which is present in utero. No other changes were observed (FIG. 7B). In the brain samples from T-Empty-NP treatment for both 5 and 10 mg/kg, NT-Empty-NP at 10 mg/kg, and saline treatment had mild meningeal and parenchymal congestion. A pathologist's professional opinion indicated this mild congestion in the brain is a nonspecific finding and likely related to euthanasia.

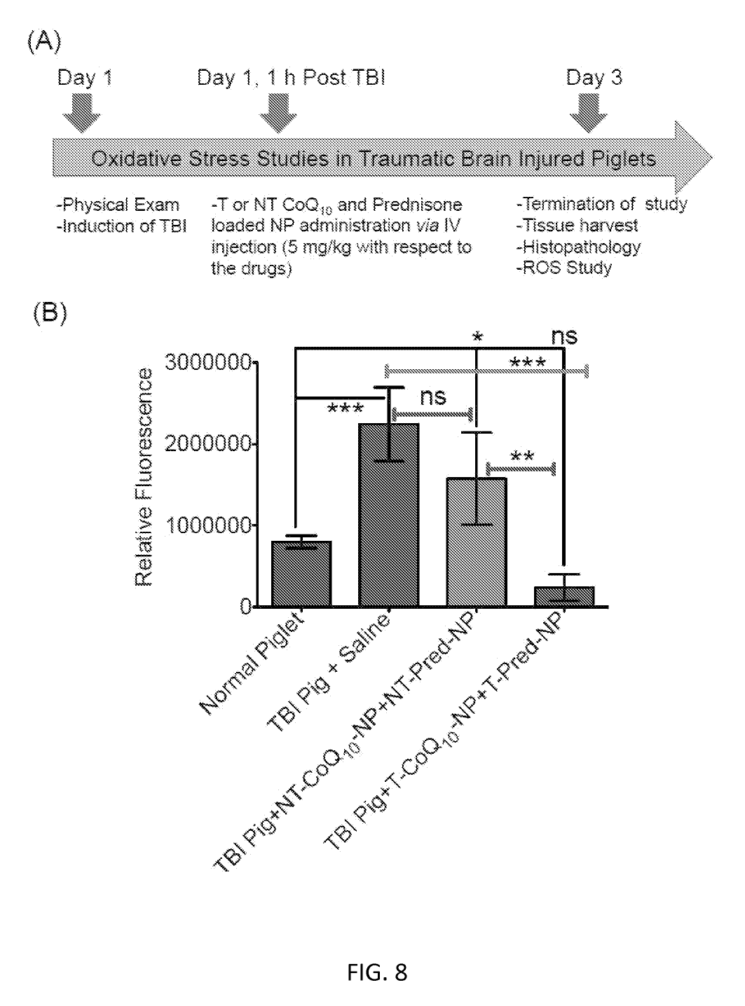

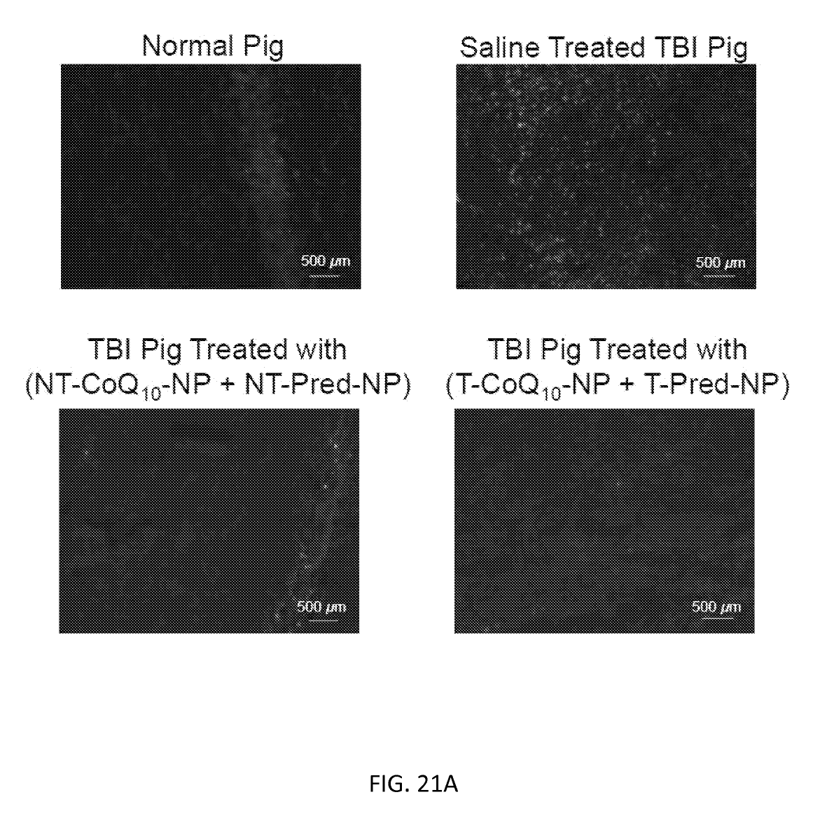

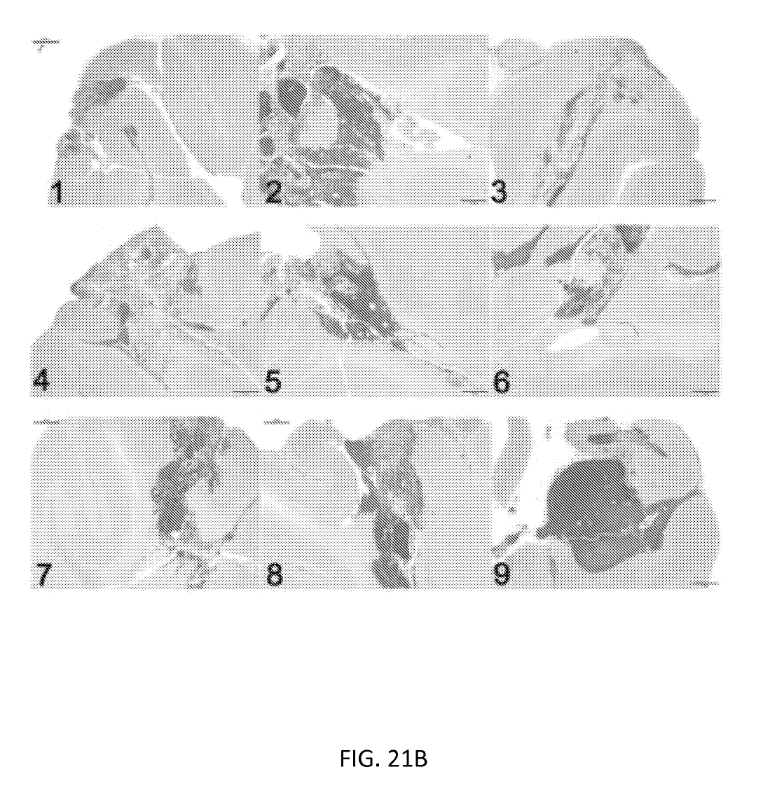

The ability of a combination of T-CoQ10-NPs and T-Pred-NPs to show reduction in oxidative stress under in vivo settings was studied in American Landrace piglets (4 weeks old, 3 per group). TBI was induced in all the 9 piglets and after 1 h a mixture of T-CoQ₁₀-NPs (5 mg/kg with respect to CoQ₁₀) and T-Pred-NP (5 mg/kg with respect to prednisone) in 10 mL of nanopure water; a mixture of NT-CoQ10-NPs (5 mg/kg with respect to CoQ10) and NT-Pred-NP (5 mg/kg with respect to prednisone) in 10 mL of nanopure water, or saline were administered via intravenous injection (FIG. 8A). Sizes and zeta potential of the NPs used in the in vivo study are given in Table 5. After 48 h, piglets were anesthetized and the brain samples were collected. The effect of the combination of T-CoQ10-NPs and T-Pred-NPs treatment on TBI induced oxidative damage was investigated. Analyses of the ROS levels in the injured ipsilateral cortex demonstrated an elevated ROS levels in the saline treated groups compared to normal piglets (FIG. 8B, FIG. 21A). Treatment with a cocktail containing T-CoQ₁₀-NPs and T-Pred-NPs attenuated the increased ROS levels in TBI-injured animals to a greater extent compared to a combination of the corresponding NT-NPs (FIG. 8B). The histological changes found from hematoxylin and eosin staining was similar across saline and T-NP treated groups indicating no toxicity in the T-NP treated animals (FIG. 21B). Necrosis and hemorrhage was present at the lesion site. Degenerate neutrophils and a small number of macrophages infiltrated the lesion site, and the endothelium in the necrotic area and surrounding neuroparenchyma was very reactive. There was also edema accumulation within the hemisphere ipsilateral to injury. The ability of a combination of brain penetrating T-CoQ₁₀-NPs and T-Pred-NPs at inhibiting oxidative stress in a TBI pi model without any apparent toxicity is a critical observation of the current work.

This study provides a potential nanomedicine platform for combined neuroprotectant-stem cell therapy after TBI. The mitochondria targeted lipophilic NPs can locally deliver a combination of anti-inflammatory and antioxidant agent in a controlled release fashion and the subsequent application of NSCs has the potential to repair the damaged tissue. The distribution of the T-NPs in the lipophilic white matter of the brain which is rich in inflammation and oxidative stress during injury provides an important means to deliver therapeutic doses locally and simultaneously to reduce the problem of systemic toxicity common to intravenously administered therapeutic agents with limited ability to cross the BBB. The targeted NPs are simple in composition which will be extremely beneficial for clinical translation and constructed from a well characterized biodegradable targeting moiety appended polymer and have the potential to encapsulate variety of hydrophobic drugs. Although this BBB penetrating biodegradable mitochondria targeted NPs were evaluated for possible use in TBI, this technology can be tailored for plethora of central nervous system diseases such as neurodegenerative disorders like Parkinson disease or Huntington disease, diseases with localized cerebral dysfunction, such as stroke.

TABLE 2
Characterization of T and NT-NPs

| | | | Polydis- | | |
|---|---|---|---|---|---|
| | | Zeta | persity | | |
| | Z_{Average} | Potential | index | % | |
| | (nm) | (mV) | (PDI) | Loading | % EE |
| T-Empty- | 52.6 ± 1.3 | 34.0 ± 0.7 | 0.256 | — | — |
| NPs | | | | | |
| NT-Empty- | 52.7 ± 0.6 | −25.1 ± 1.5 | 0.244 | — | — |
| NP | | | | | |
| T-Pred-NP | 55.8 ± 2.6 | 27.3 ± 3.6 | 0.133 | 18.4 ± 0.8 | 61.6 ± 2.5 |
| NT-Pred-NP | 53.5 ± 1.4 | −17.4 ± 2.1 | 0.199 | 18.8 ± 1.7 | 62.6 ± 5.7 |
| T-CoQ₁₀-NP | 67.1 ± 1.2 | 52.4 ± 3.1 | 0.148 | 24.9 ± 2.6 | 83.7 ± 8.7 |
| NT-CoQ₁₀- | 65.3 ± 0.6 | −16.3 ± 0.5 | 0.132 | 23.5 ± 2.1 | 78.8 ± 6.9 |
| NP | | | | | |

% Feed of Prednisone and CoQ₁₀ used was 30%

TABLE 3
Characterization of QD loaded T
and NT-NPs used in animal studies

| | | | |
|---|---|---|---|
| | Zeta | | |
| Z_{Average} | Potential | | |
| (nm) | (mV) | PDI | QD Content |

PK and bioD in Normal Pig

| | | | | |
|---|---|---|---|---|
| NT-QD-NP | 53.0 ± 0.3 | −13.7 ± 1.1 | 0.183 ± 0.010 | 0.150 mg Cd/ |
| | | | | mg polymer |
| T-QD-NP | 50.5 ± 0.8 | 42.4 ± 1.9 | 0.447 ± 0.027 | 0.127 mg Cd/ |
| | | | | mg polymer |

PK and bioD in TBI Pig

| | | | | |
|---|---|---|---|---|
| NT-QD-NP | 49.3 ± 1.5 | −16.0 ± 1.3 | 0.169 ± 0.025 | 0.124 mg Cd/ |
| | | | | mg polymer |
| T-QD-NP | 60.5 ± 7.5 | 43.4 ± 2.0 | 0.295 ± 0.089 | 0.092 mg Cd/ |
| | | | | mg polymer |

TABLE 4
PK Profiles of T and NT-NPs in TBI Pig Model

| | T-NPs | NT-NPs |
|---|---|---|
| Cd Does (mg/kg) | 0.889 | 1.05 |
| AUC_{[0-24 h]} (ng · h/ml) | 43,449 ± 742 | 32,137 ± 5,315 |
| Cₘₐₓ (ng/ml) | 4,143 ± 1,228 | 4,324 ± 678 |
| V_{d} (ml/kg) | 226.0 ± 67.0 | 246.0 ± 38.6 |
| C_{L} (ml/h) | 85.3 ± 15.8 | 156.8 ± 35.2 |
| T_{1/2} (h) | 9.6 ± 4.51 | 5.68 ± 2.13 |

Least-squares fit to model: C = A*exp(−k₁*t)

TABLE 5
Characterization of T and NT-CoQ₁₀ and Prednisone loaded
NPs used in efficacy study using TBI pig model

| | | Zeta | | % |
|---|---|---|---|---|
| | Z_{Average} | Potential | Polydispersity | Load- |
| | (nm) | (mV) | index (PDI) | ing |
| T- CoQ₁₀-NP | 46.24 ± 0.45 | 43.4 ± 0.93 | 0.084 ± 0.023 | 23.44 |
| T-Pred-NP | 44.61 ± 0.45 | 44.6 ± 1.46 | 0.101 ± 0.040 | 4.69 |
| NT-CoQ₁₀-NP | 51.46 ± 1.12 | −17.0 ± 0.99 | 0.164 ± 0.098 | 8.68 |
| NT-Pred-NP | 47.16 ± 0.089 | −19.9 ± 1.8 | 0.093 ± 0.007 | 4.76 |

### Materials and Methods.

### Animals.

American Landrace piglets (4 weeks old) were obtained from the University of Georgia Swine Farm and handled in accordance with “The Guide for the Care and Use of Laboratory Animals” of American Association for Accreditation of Laboratory Animal Care (AAALAC), Animal Welfare Act (AWA), and other applicable federal and state guidelines. All animal work presented here was approved by Institutional Animal Care and Use Committee (IACUC) of University of Georgia.

### Statistics.

All data were expressed as mean±S.D (standard deviation). Statistical analysis were performed using GraphPad Prism® software v. 5.00. Comparisons between two values were performed using an unpaired Student t test. A one-way ANOVA with a post-hocTukey test was used to identify significant differences among the groups.

### Materials and Instrumentations.

All chemicals were received and used without further purification unless otherwise noted. Dimethylaminopyridine (DMAP), KCl, N-hydroxysuccinimide (NHS), triethylamine, 5-bromopentanoic acid, N,N′-dicyclohexylcarbodiimide (DCC), hydrogen peroxide solution (30 wt. % in H₂O), (3-(4,5-dimethylthiazol-2-yl)-2,5-diphenyltetrazolium bromide (MTT), CoQ₁₀ (Product number C9538), and prednisone (product number P6254) were purchased from Sigma-Aldrich. Carboxy terminated PLGA (dL/g, 0.15 to 0.25) was procured from Lactel and OHPEG-OH of molecular weight 3350 was purchased from Sigma Aldrich. TPP was purchased from Sigma Aldrich. Bicinchoninic acid (BCA) protein assay kit (Pierce 23227) was purchased from Thermo Scientific. Sodium chloride, magnesium chloride, sucrose, potassium chloride, and ethylyenediaminetetraacetic acid (EDTA) were purchased from J. T. Baker. Slide-A-Lyzer MINI Dialysis Units (catalog number 69572) were purchased from Thermo Scientific. Human induced pluripotent NSCs were purchased from GlobalStem. Matrigel was purchased from BD Bioscience. Accutase was purchased from Innovative Cell Technologies. NSC expansion media reagents were purchased from Life Technologies with the exception of basic fibroblast growth factor (bFGF), which was purchased from R&D Systems. 2′7′-Dichlorofluorescin diacetate (DCF-DA) for reactive oxygen species detection was purchased from Sigma Aldrich. Swine specific enzyme-linked immunosorbent assay (ELISA) kits were purchased from Life Technologies and absorbance was read on a FlexStation Plate Reader from Molecular Devices. Human specific ELISA kits were purchased from R&D Systems. MitoTracker® Green was purchased from Invitrogen. Qdot® 705 ITK™ Amino (PEG) Quantum Dots (catalog number Q21561MP) and prolong Gold with DAPI were purchased from Life Technologies. Seahorse XF24 well plates and cartridges were purchased from Seahorse Bioscience. Anti glial fibrillary acidic protein (Anti-GFAP) antibody (Catalog number: ab4674) and anti-CD31 antibody (Catalog number: ab28364) were purchased from Abcam. Oligodendrocyte transcription factor (Olig2) antibody was purchased from GennTex (Catalog number: GTX62440). Anti-NeuN purified antibody was procured from EMD Millipore (Catalog number: ABN90P). Secondary antibodies, Alexa Fluor mouse 488-A11001, Alexa Fluor guinea pig 488-A 11073, Alexa Fluor rabbit 488-A111034 were procured from Invitrogen. Chicken 647-SAB4600179 secondary antibody was purchased from Sigma. Natural donkey serum (NDS) was obtained from Millipore (Catalog No. S30-100ML).

Distilled water was purified by passage through a Millipore Milli-Q Biocel water purification system (18.2 MΩ) containing a 0.22 μm filter. Cells were counted using Countess® Automated cell counter procured from Invitrogen. DLS measurements were carried out using a Malvern Zetasizer Nano ZS system. Optical measurements were carried out on a NanoDrop 2000 spectrophotometer. TEM images were acquired using a Philips/FEI Technai 20 microscope. Inductively coupled plasma mass spectrometry (ICP-MS) studies were performed on a VG PlasmaQuad 3 ICP mass spectrometer. Plate reader analyses were performed on a Bio-Tek Synergy HT microplate reader. Antioxidative stress assays were carried out using a Seahorse XF24 analyzer (Seahorse Biosciences, North Billerica, Mass., USA). Fluorescence imaging of brain samples was carried out on a Xenogen IVIS® Lumina system. Confocal images of brain tissue samples were captured on a Nikon AIR confocal microscope. Serum clinical chemistry analyses were performed on Hitachi P-Modular system, Roche Diagnostics. Hematology analyses were performed on a HemaTrue Hematology Analyzer, Heska.

### Cell Culture.

Human induced pluripotent stem cell-derived neural stem cells (HIP™ hNSC BC1, GlobalStem®, Rockville, Md.) used for the in vitro portion of this study. NSCs were maintained on matrigel-coated tissue culture dishes in neural stem cell media composed of neural basal medium, 2% B-27 supplement, 1% non-essential amino acids, 2 mM L-alanine/L-glutamine, 1% penicillin/streptomycin, and 20 ng/mL bFGF. The media was changed every other day. When NSCs reached confluence (approximately every 4 to 5 days), cells were enzymatically passaged using Accutase and removed from the dish using a cell scrapper. Cells were split at a 1:4 ratio. To prepare NSCs for MTT and ROS Reduction assays, NSCs were plated in matrigel-coated 96-well dishes at a ratio of 40,000 cells per well in neural stem cell media.

### Synthesis of PLGA-b-PEG-OH and PLGA-b-PEG-TPP.

The non targeted PLGA-b-PEG-OH and targeted PLGA-b-PEG-TPP polymers were synthesized and characterized by following methods previously reported in Marrache, S. & Dhar, S. Engineering of blended nanoparticle platform for delivery of mitochondria-acting therapeutics. Proc Natl Acad Sci USA 109, 16288-16293, doi:10.1073/pnas.1210096109 (2012).

### Synthesis of PLGA-PEG-QD.

Conjugation of PLGA to PEG-QD was carried out using a slight modification based on a previously reported method (Marrache & Dhar (2012), Proc Natl Acad Sci USA 109, 16288-16293). PLGA-COOH of inherent viscosity 0.15-0.25 (1 mg, ˜0.16 μmol) was dissolved in 2 mL DMF. To activate the carboxyl moiety for conjugation, EDC (10 mg, 52 μmol) and NHS (10 mg, 86.9 μmol) were added, and the mixture was stirred at room temperature for 5 h. To this, QDot® 705 ITK Amino (PEG) quantum dots (250 μL, 2 nmol) added dropwise slowly to ensure no visible precipitation of PLGA happens. The reaction mixture was stirred overnight at room temperature. The PLGA-PEG-QD was isolated by repeated washing and centrifugations (14,000 rpm, 1 h) using DMF. The final PLGA-PEG-QD pellet was resuspended in 250 μL of DMF and kept at 4° C. until further use.

### T and NT-QD NP Construction.

T and NT NPs containing QD were synthesized by a nanoprecipitation method. PLGA-b-PEG-OH or PLGA-b-PEG-TPP was dissolved in dimethyformamide (DMF) at a concentration of 50 mg/mL. A 100 μL solution of the polymer was mixed with PLGA-PEG-QDs (10 μL, 8 μM solution in DMF) and diluted with DMF to a final polymer concentration of 5 mg/mL. This mixture was added drop-wise to nanopure water with constant stirring at room temperature. The NPs were stirred for 2 h at room temperature in a fume hood. Organic solvent was removed by washing three times using a 100 kDa cut-off amicon filtration membrane at 3000 rpm and 4° C. The NPs were resuspended in 1 mL nanopure water at a concentration of 5 mg/mL and stored at 4° C. until further use. DLS measurements were carried to determine size, PDI, and zeta potential.

### T and NT-Pred-NP Synthesis.

Prednisone loaded T and NT NPs were synthesized from PLGA-b-PEG-TPP or PLGA-b-PEG-OH by a nanoprecipitation method. PLGA-b-PEG-OH or PLGA-b-PEGTPP in (50 mg/mL in DMF) was mixed with a predefined amount of prednisone (10 mg/mL in DMF) and diluted with DMF to a final polymer concentration of 5 mg/mL. In all our studies, we used 30% feed of prednisone with respect to polymer weight. This mixture was added drop-wise to nanopure water with constant stirring. The NPs were stirred for 2 h at room temperature in a fume hood. Organic solvent was removed by washing three times using a 100 kDa cut-off amicon filtration membrane using 3000 rpm at 4° C. The NPs were resuspended in 1 mL nanopure water at a concentration of 5 mg/mL stored at 4° C. until further use. DLS measurements were carried to determine size, PDI, and zeta potential using NP suspension of ˜0.25 mg/mL concentration. Percent prednisone loading and % EE were determined by dissolving the polymeric core in 0.1 mM NaOH for 1 h at room temperature. The resulting solution was further dissolved in a 50:50 mixture of DMF:H₂O and prednisone was quantifies using HPLC (wavelength used: 261 nm, Zorbax 300SB C18 column, 50:50 acetonitrile with 0.1% trifluoroacetic acid (TFA):isopropanol, 1 mL/min flow rate, using a retention time of 11.4 min).

### Synthesis of T and NT-CoQ10-NPs.

PLGA-b-PEG-OH or PLGA-b-PEG-TPP in (50 mg/mL in DMF) was mixed with 150 μL of 10 mg/mL solution of CoQ₁₀ in DMF and diluted with DMF to a final polymer concentration of 5 mg/mL. The CoQ₁₀ feed was 30% with respect to the polymer. This mixture was added drop-wise to nanopure water with constant stirring. The NPs were stirred for 2 h at room temperature in a fume hood. Organic solvent was removed by washing three times using a 100 kDa cut-off amicon filtration membrane with 3000 rpm at 4° C. The NPs were resuspended in nanopure water (1 mL) at a concentration of 5 mg/mL and stored at 4° C. until further use. DLS measurements were carried to determine size, PDI, and zeta potential (0.25 mg/mL, each measurement was an average of three individual measurements). Percent CoQ₁₀ loading and % EE were determined by dissolving the polymeric core in in 0.1 mM NaOH for 1 h at room temperature. The resulting solution was further dissolved in a 50:50 mixture of DMF:H2O and prednisone was quantifies using HPLC (wavelength used: 329 nm, Zorbax 300SB C18 column, 50:50 acetonitrile with 0.1% TFA:isopropanol, 1 mL/min flow rate, using a retention time of 7.2 min).

Release of Prednisone and CoQ10 from T and NT-NPs.

The release kinetics of prednisone and CoQ10 from the T and NT NPs were analyzed by subjecting these NPs to dialysis against 1×PBS (pH 7.4, 4 L) at 37° C. The NPs (100 μL) were added to a Slide-a-lyzer mini dialysis unit and placed in the PBS bath with gentle shaking. PBS was changed every 12 h. At various time points two dialysis units were removed. The amount of prednisone or CoQ₁₀ remained was determined by dissolving the polymeric core as described before and quantifying the amount of prednisone or CoQ₁₀ released using HPLC using conditions described above.

### Antioxidative Properties of T and NT NPs by Seahorse Analyzer.

T-CoQ₁₀-NPs, NT-CoQ₁₀-NPs, and free CoQ₁₀ were tested for their ability to reduce oxidative stress in NSCs. The cells were plated at a concentration of 30,000 cells/well on each well of Seahorse XF24 well plate and allowed to grow overnight. Each well was first coated with 50 μL matrigel before cells were plated. The media was changed and the cells were washed with Seahorse basal media and the media was replaced with seahorse basal media. The cells were monitored for changes in oxygen consumption rate (OCR) with respect to time. A basal reading was acquired for 45 min and then H₂O₂(10 μM) was injected. The OCR was monitored for 90 min and T-CoQ₁₀-NPs (1 μM with respect to CoQ₁₀), NT-CoQ₁₀-NPs (1 μM with respect to CoQ₁₀), and free CoQ₁₀ (1 μM) were injected. The OCR levels were then further measured for 4.5 h. As a control, cells were also monitored with no treatment or only treatment of H₂O₂(10 μM).

### Anti-Inflammatory Properties of NPs in Stem Cells.

NSCs were plated on each well of a 12-well plate at a density of 1×107 per well and allowed to grow overnight (NSC basal media in 200 μL). LPS (100 ng/mL) was added to the NSCs and incubated for 36 h. T-CoQ₁₀-NPs, NT-CoQ₁₀-NPs, T-Prednisone-NPs, NT-Prednisone-NPs, T-CoQ₁₀-NPs+T-Prednisone-NPs, NT-CoQ₁₀-NPs+NTPrednisone-NPs, free CoQ₁₀, free prednisone, or free prednisone+free CoQ₁₀ (For NPs or free formulations: 1 μM with respect to CoQ₁₀ or Prednisone). ELISA was performed on the supernatants against the cytokines interleukin (IL)-6, IL-10, and TNF-α, IL-12, and IL-4 according to the methods reported by us. Briefly, antibody coated plates were blocked with 10% FBS in PBS for 1 h at room temperature followed by 3 washes with wash buffer. NSC supernatants were incubated on the plates for 2 h at room temperature. This was immediately followed by washings and sequential incubations with the cytokine-biotin conjugate and streptavidin working solution. Finally, the substrate reagent containing 3,3′,5,5′-tetramethylbenzidine (100 μL) was added to each well, incubated for 15 min, the reaction was stopped by adding 50 μL H₂SO₄ (0.1 M). The absorbance was recorded at 450 nm using a BioTek Synergy HT well plate reader.

### Cytotoxicity of COQ10 and Prednisone NPs in NSCs.

The cytotoxic behavior of all the NPs was evaluated by using the MTT assay against NSCs. Cells (40,000 cells/well) were seeded on a 96-well plate in 100 μL of NSC basal complete medium and incubated for 24 h. The cells were treated with NPs at varying concentrations (with respect to prednisone and CoQ10) and incubated at 37° C. The medium was changed after 12 h, and the cells were incubated for additional 60 h. The cells were then treated with 20 μL of MTT (5 mg/mL in PBS) for 5 h. The medium was removed, the cells were lysed with 100 μL of DMSO, and the absorbance of the purple formazan was recorded at 550 nm using a Bio-Tek Synergy HT microplate reader. Each well was performed in triplicate and a background reading was recorded at 800 nm.

### BioD and PK of NPs in Pig.

American Landrace piglets (4 weeks old, 3 per group) were anesthetized using isofluorane. T-QD-NPs (2.5 mg/kg with respect to NP and 0.46 mg/kg with respect to Cd), NT-QD-NPs (2.5 mg/kg with respect to NP and 0.62 mg/kg with respect to Cd), and saline were administered via intravenous catheter placed into an ear vein. Blood samples were collected in heparinized tubes at 0, 2, 4, 6, 8, and 24 h post-injection and stored at 4° C. until use. Blood samples were centrifuged at 2000 rpm for 20 min at 4° C. in order to collect plasma. The percentage of QD from NPs was calculated by taking into consideration that blood constitutes 3.5% of body weight and plasma constitutes 55% of blood volume for pig. The amount of Cd from the QD was calculated in the blood plasma by ICP-MS. After 24 h post-surgery, piglets were deeply anesthetized using 5% vaporized isoflurane with oxygen utilizing a surgical mask and then euthanized via CO₂ inhalation. After euthanasia, the piglets were decapitated and the brain was removed and stored at −80° C. For bioD studies, the heart, lungs, kidneys, liver, and spleen were removed and stored at −80° C. The overall bioD was calculated by analyzing the amount of Cd in each organ by ICP-MS. The brain samples were also imaged by IVIS using Cy5.5 emission and 500 nm excitation with an exposure time of 1 sec. Brains were then sectioned into 5 mm coronal sections and imaged by IVIS. Before analysis, the organs were dissolved with nitric acid (typically 1 g of tissue/10 mL of acid) for 24 h with gentle heating and shaking. The calculations for AUC, Cₘₐₓ, Tₘₐₓ, and C_{L} (t=0) were performed in the GraphPad Prism (Version 5.01). PK parameters were determined by fitting the data using a one-compartmental model equation.

### Cryosectioning of Pig Brains for Confocal Imaging.

Liver samples as well as grey and white matter samples from brain were isolated. Samples were fixed in 4% paraformaldehyde for 5 h before being cryoprotected in 30% sucrose. Samples were then embedded in Optimal Cutting Temperature (OCT) compound and stored at −80° C. before being cryosectioned at 5 μm using a cryostat. The sections were then stained with MitoTracker green (500 nM) for 45 min at room temperature. The sections were then washed 3 times with 1×PBS and 5 times with nanopure H₂O. The sections were then suspended in anti-fade reagent and covered with a coverslip for imaging. For sections stained with DAPI, slides were mounted in Prolong Gold with DAPI before being coverslipped.

### Induction of TBI in Pig.

Five 4-week old domestic Landrace piglets underwent surgery. Piglets were anesthetized using 5% vaporized isoflurane with oxygen utilizing a surgical mask and heart rate, respiration rate, and body temperature were continuously monitored during surgery. After routine skin sterilization, a skin incision was made at midline at the top of the cranium. Using a trephine, a craniotomy 7 mm in diameter was performed in the frontal bone at the anterior junction of the left coronal and metopic sutures. The dura was left intact and care was taken to avoid trauma to the cortical surface. A sterile surgical blade was inserted vertically into the left frontal lobe at a depth of 15 mm to the dura surface and turned 360 degrees before removal. The exposed cortical surface was covered with sterile bone wax, and the skin incision was closed with surgical staples. The piglets were allowed to recover from general anesthesia and were monitored until ambulatory.

### Histopathology.

Coronal brain sections were fixed in 10% neutral-buffered formalin, routinely processed, embedded in paraffin, sectioned approximately 5 μm, mounted on glass slides, and stained with hematoxylin and eosin.

### ROS Detection.

Brain tissue samples were removed from the injury site and snap frozen in liquid nitrogen and stored at −80° C. Tissue samples were cryosectioned at 10 μm thickness and immediately fixed in 4% paraformaldehyde on glass microscopy slides. ROS detection was performed by applying 2′,7′-dichlorofluorescin diacetate (Sigma-Aldrich, Cat#D6883) and incubating at 37° C. for 30 min. Slides were washed once with phosphate buffered saline before imaging. Imaging was performed on a Nikon TE2000-S microscope equipped with a QImaging Retiga 2000R camera with an exposure time of 1 sec using a EXFO X-Cite 120 bulb for GFP filter. Images were randomly taken across tissue sections and total fluorescence of the images was measured via ImageJ. 5 pictures were taken per pig, 3 pigs per treatment group.

### ELISA on Brain Sample.

Brain tissue samples were removed from the injury site and snap frozen in liquid nitrogen and stored at −80° C. Tissue samples were homogenized in cold radioimmunoprecipitation assay (RIPA) lysis buffer (Amresco, Cat# N653) with a protease inhibitor cocktail (Amresco, Cat# M222). Tissue homogenate aliquots were microcentrifuged at 13.500 rpm for 45 min. The resulting supernatant was stored at −80° C. until use. Protein levels of TNF-α and IFN-γ were quantified using ELISA employing pig-specific assay systems (Invitrogen Cat# KSC3011 and KSC0081, respectively). Tissue lysate was diluted 1:10 in standard diluent buffer (provided in the kit) and the manufacturer's instructions were followed thereafter. Each sample was run in triplicate. The absorbance was measured at 450 nm using a Flexstation plate reader. Data was analyzed by running a four parameter logistic utilizing SigmaPlot 12.5 software.

### BioD and PK of NPs in TBI Pig Model.

American Landrace piglets (4 weeks old, 2 per group) were anesthetized using isofluorane and the TBI was induced as mentioned above. After 5 h, T-QD-NPs (1.5 mL, 2.33 mg/kg with respect to NP and 0.889 mg/kg with respect to Cd), NT-QDNPs (1.5 mL suspension, 2.33 mg/kg with respect to NP and 1.05 mg/kg with respect to Cd), and saline were administered via intravenous catheter placed into an ear vein and nanoparticle solution was administered. Blood samples were collected in heparinized tubes at 0, 2, 4, 6, 8, and 24 h post-injection via orbital sinus bleed and stored at 4° C. until use. Blood samples were centrifuged at 2000 rpm for 20 minutes at 4° C. in order to collect plasma. The percentage of QD from NPs was calculated by taking into consideration that blood constitutes 6.5% of body weight and plasma constitutes 55% of blood volume for pig. The amount of Cd from the QD was calculated in the blood plasma by ICP-MS. After 24 h post-surgery, piglets were deeply anesthetized using 5% vaporized isoflurane with oxygen utilizing a surgical mask and then euthanized via CO₂ inhalation. After euthanasia, the piglets were decapitated and the brain was removed and stored at −80° C. The injured area of the brain was sectioned into 5 mm coronal sections for analysis. For bioD studies, the heart, lungs, kidneys, liver, and spleen were removed and stored at −80° C. The overall bioD was calculated by analyzing the amount of Cd in each organ by ICP-MS. The brain samples were also imaged by IVIS using 570 nm excitation and Cy5.5 emission filters with an exposure time of 1 sec. Brains were then sectioned into 5 mm coronal sections and imaged by IVIS. Before analysis, the organs and feces were dissolved with PerkinElmer solvable (Product number: 6NE9100) for at least 24 h with gentle heating and shaking. The calculations for AUC, Cₘₐₓ, Tₘₐₓ, and C_{L} (t=0) were performed in the GraphPad Prism (Version 5.01). PK parameters were determined by fitting the data using a one-compartmental model equation.

### Isolation and Preparation of Grey and White Matter for ICP-MS.

After coronal brain sections were imaged on IVIS, the grey and white matters were dissected manually using a scalpel blade. Approximately 200 mg tissue samples of both grey and white matter from each animal were put into microtubes and stored at −20° C. until use. After isolation, the grey and white matter samples from each pig (˜200 mg each) was placed in an Eppendorf tube. The tissue was dissolved with concentrated nitric acid (1 mL) with heating at 50° C. and gentle shaking for 4 h. The amount of Cd was quantified by ICPMS.

### Time Dependent BioD Studies in Mice.

Male (C57BL/6) mice (30 g) were purchased form Charles River, USA, and work was performed under aseptic condition. The animals were anesthetized in presence with isoflurence (2%) with equally amount of oxygen. T-QD-NPs were injected as a dose of 20 mg/kg. T-QD-NPs in this study had average diameter of 69.2±1.1 nm and zeta potential of 31.5±0.8 mV. After 12, 24, or 48 h of injection, surgery was performed under aseptic condition and all the major organs were isolated and imaged immediately using IVIS with 500 nm excitation and Cy5.5 emission filters with an exposure time of 2 sec. After imaging, brain samples were fixed in 4% paraformaldehyde for 48 h at 4° C. before being cryoprotected in 30% sucrose. Samples were then embedded in OCT compound and stored at −80° C. before being cryosectioned at 5 μm using a cryostat. The sections were then stained with different antibodies for immunofluorescence studies.

Immunostaining of tissue sections were performed using antibody treatments against different types of brain cell markers: NeuN for neuronal nuclei, CD-31 for normal endothelial marker, olig2 for oligodendrocytes, and GFAP for astrocytes. The brain sections were washed thoroughly 2-3 times with PBS (1×) without disturbing the section regions and then blocked with 10% NDS in PBS (1×) containing 0.3% Triton-X for 1 h at room temperature. The tissues sections were incubated overnight at 4° C. with the primary antibodies using following dilutions: anti-NeuN antibody (1:500 dilution), anti-CD-31 antibody (1:50 dilution), anti-olig2 (1:250 dilution), and anti-GFAP (1:500 dilution) in a humidified chamber. After overnight incubation, the sections were washed 3 times with PBS (1×). The sections were incubated for 1.5 h with the following secondary antibody: Alexa Fluor guinea pig 488-A11073 for NeuN, Alexa Fluor rabbit 488-A111034 (invitrogen) for CD31 and Olig2, chicken 647-SAB4600179 for GFAP. The sections were washed 3 times with PBS, mounted, covered with coverslip, and observed under a confocal microscope.

### Dose Dependent 14-Day Toxicity Study in Pigets.

Toxicity studies were carried out in American Landrace piglets (4 weeks old, 2 per group) at 5 mg/kg and 10 mg/kg of total NP dose of T-Empty-NPs and NT-Empty-NPs for 14 days. On day 0, a single intravenous injection of T-Empty-NPs and NT-Empty-NPs at 5 mg/kg and 10 mg/kg with respect to total NP was diluted to 1 mL (for 5 mg/kg) or 2 mL (for 10 mg/kg) and given intravenously via the ear vein. T-Empty-NPs in this study had average diameter of 47.4±2.9 nm and zeta potential of 46.2±0.4 mV. NT-Empty-NPs in this study had average diameter of 72.8±1.7 nm and zeta potential of −16.1±0.4 mV. Post-injection, the animals were monitored for 14 days. Blood was collected on day 7 and day 14 via venipuncture and stored in anti-coagulant coated tubes for complete blood count analysis. Blood samples were also collected in separate tubes and serum was isolated for serum chemistry panel analysis. The animals were euthanized on day 14 and spleen, kidneys, liver, heart, lungs, and brain were removed and cleaned of excess material. Liver and brain samples were fixed in 10% buffered formalin. A section of well-fixed liver and unilateral sections of brain were sampled as described by Bolon et al. 4 for sampling and processing the nervous system during nonclinical general toxicity studies. The samples were routinely processed and embedded in paraffin, and then 4 μm sections were stained with hematoxylin (H) and eosin (E).

### Anti-Inflammatory and Anti-Oxidative Properties of T- and NT-CoQ10 and Prednisone NPs in TBI Pig Model.

American Landrace piglets (4 weeks old, 3 per group) were anesthetized using isofluorane and the TBI was induced as mentioned above. After 1 h, T-CoQ₁₀-NPs (5 mg/kg with respect to CoQ10) and T-Pred-NP (5 mg/kg with respect to Prednisone) mixing together in 10 mL of nanopure water and NTCoQ₁₀-NPs (5 mg/kg with respect to CoQ₁₀); NT-Pred-NP (5 mg/kg with respect to Prednisone) mixing together in 10 mL of nanopure water; and saline were administered via intravenous catheter placed into an ear vein over 10 min. After 48 h, piglets were deeply anesthetized using 5% vaporized isoflurane with oxygen utilizing a surgical mask and then euthanized via CO₂ inhalation. After euthanasia, the piglets were decapitated and the brain was removed and sectioned. Brain samples (˜200 mg in size) were isolated, snap frozen, and stored at −80° C. for ROS detection. Whole coronal brain sections were fixed in 10% neutral-buffered formalin for histological analysis. Brain tissue samples were processed following methods described above to perform ROS detection using 2′,7′-dichlorofluorescin diacetate. Coronal brain sections that were fixed in 10% neutral-buffered formalin were routinely processed, embedded in paraffin, sectioned approximately 5 μm, mounted on glass slides, and stained with hematoxylin and eosin for histological analysis.

Thus, embodiments of THERAPEUTIC NANOPARTICLES FOR ACCUMULATION IN THE BRAIN are disclosed. One skilled in the art will appreciate that the nanoparticles and methods described herein can be practiced with embodiments other than those disclosed. The disclosed embodiments are presented for purposes of illustration and not limitation.

1. A nanoparticle, comprising:
a mitochondria targeting moiety;
and one or more of (i) an antioxidant agent; (ii) an anti-inflammatory agent, and (iii) imaging agent.

2. A nanoparticle according to claim 1, wherein the nanoparticle comprises an anti-oxidant and an anti-inflammatory agent.

3. A nanoparticle according to claim 1, wherein the anti-oxidant is CoQ₁₀.

4. A nanoparticle according to claim 1, wherein the anti-inflammatory agent is a steroidal anti-inflammatory agent.

5. A nanoparticle according to claim 1, wherein the anti-inflammatory agent is prednisone.

6. (canceled)

7. A nanoparticle according to claim 1, wherein the nanoparticle comprises the imaging agent, wherein the imaging agent is attached to a targeting molecule, wherein the targeting molecule interacts with a therapeutically or diagnostically relevant molecule.

8-9. (canceled)

10. A nanoparticle according to claim 1, wherein the mitochondrial targeting moiety comprises a hydrophobic delocalized cationic moiety.

11. (canceled)

12. A nanoparticle according to claim 1, wherein the mitochondrial targeting moiety comprises a triphenyl phosophonium (TPP) moiety or a derivative thereof.

13. A nanoparticle according to claim 1, further comprising:
a hydrophobic nanoparticle core; and
a hydrophilic layer surrounding the core.

14. A nanoparticle according to claim 13, wherein the hydrophilic layer comprises PEG.

15-18. (canceled)

19. A nanoparticle according to claim 13, wherein the hydrophobic nanoparticle core comprises a polymer selected from polylactic acid (PLA), polycaprolactone (PCL), polyglycolic acid (PGA), and polylactic-co-glycolic acid (PLGA).

20-23. (canceled)

24. A method comprising administering a nanoparticle according to claim 1 to a subject.

25. A method for treating a patient at risk f or suffering from damaged neural tissue, comprising administering a nanoparticle according to claim 1 to the patient.

26. A method according to claim 25, further comprising identifying the patient as a patient at risk of or suffering from damaged neural tissue.

27. A method for treating a patient at risk f or suffering from traumatic brain injury, comprising administering a nanoparticle according to claim 1 to the patient.

28. A method according to claim 27, further comprising identifying the patient as a patient at risk of or suffering from traumatic brain injury.

29. A method for treating a patient at risk of or suffering from a brain related disease, comprising administering a nanoparticle according to claim 1 to the patient.

30. A method according to claim 29, further comprising identifying the patient as a patient at risk of or suffering from the brain related disease.

31. A method according to claim 24, wherein administering the nanoparticle comprises systemically administering the nanoparticle.
